(19) United States
(12) Patent Application Publication
(10) Pub. No.: US 2026/0091172 A1
(43) Pub. Date: Apr. 2, 2026

(54) WEARABLE THERMAL SENSORS

(71) Applicant: RHAEOS, INC., EVANSTON, IL (US)

(72) Inventors: Richard Chad WEBB, Chicago, IL (US); Blake Valenti PARSONS, Chicago, IL (US); Chase CORREIA, Chicago, IL (US); Alex BURTON, Chicago, IL (US)

(73) Assignee: RHAEOS, INC., EVANSTON, IL (US)

(21) Appl. No.: 19/346,363

(22) Filed: Sep. 30, 2025

Related U.S. Application Data
(60) Provisional application No. 63/701,456, filed on Sep. 30, 2024.

(51) Int. Cl. A61M 5/168

(52) U.S. Cl. CPC A61M 5/16836; A61M 2205/18; A61M 2205/3327; A61M 2205/3368; A61M 2205/36; A61M 2205/502; A61M 2205/581; A61M 2205/582; A61M 2205/583; A61M 2205/8206; A61M 2210/04; A61M 2230/50

(57) ABSTRACT

Systems and methods for monitoring subdermal fluid are disclosed. The subdermal fluid monitoring system may include a flexible substrate, at least six temperature sensors and at least three thermal actuators supported by the flexible substrate, an adhesive substrate, a power source configured to supply power to the at least six temperature sensors and to the at least three thermal actuators; a memory storage device; and a microprocessor in communication with the memory storage device. The microprocessor may be configured to activate the power source to supply power to at least one thermal actuator to cause heating of a subdermal fluid, receive information associated with a temperature of the subdermal fluid from at least one temperature sensor; calculate an alert condition based on a set of instructions received from a memory storage device and the temperature of the subdermal fluid; and trigger an alert if the alert condition is met.

### TECHNICAL FIELD

The description herein generally relates to the field of wireless thermal sensing, and more particularly to systems and methods of monitoring peripheral intravenous catheter (PIVC) complications, including but not limited to infiltration, extravasation, phlebitis, infection, and tissue ischemia using non-invasive, wearable epidermal electronics. The device's application can be used in both a clinical setting but also extend to home use where continuous, non-invasive, monitoring is crucial for patient safety and effective delivery of intravenous (IV) fluids.

### BACKGROUND

Thermal sensors are used to monitor the temperature of objects and, when utilizing a thermal sensor which has an electrical property which varies in response to temperature change, can be implemented as part of an electrical sensing system. In some applications it may be advantageous to monitor thermal transport properties, such as conduction, convection, heat capacity, diffusivity and/or effusivity, of an object in addition to temperature. A sensor for monitoring thermal transport properties of an object can be created by combining one or more temperature sensors with one or more thermal actuators as part of a sensing system. By measuring the temperature change of an object in response to actuation by the thermal actuator, various thermal transport properties can be determined, such as thermal conductivity, heat capacity, diffusivity, and other related parameters. Different spatial locations of temperature sensors relative to the thermal actuator result in different thermal responses to thermal actuation. Some arrangements of temperature sensors and thermal actuators additionally enable measurement of convective thermal transport properties induced by the movement of fluids near to the thermal actuator.

While fluid sensors based on thermal actuation, such as hot-wire anemometers, are often desired to be in direct contact with a fluid, certain sensor arrangements enable fluid measurements through a solid media, without direct contact with the fluid. For example, such designs can enable body-mountable sensors for noninvasive measurement of biological fluids beneath the skin surface without directly contacting the biological fluid.

There are several challenges associated with making skin-mountable thermal sensors, and in particular, non-invasive thermal fluid sensors which are precise, accurate, and reliable. One of such challenges is minimizing the thermal resistance between actuators and/or sensors and the measurement object, which may be a bodily fluid beneath the skin. Some of the currently existing conventional designs employ materials between the sensors/actuators and the skin which create thermal resistance, thereby limiting the accuracy and reliability of the measurements.

Some existing sensors are mechanically rigid, which limits their ability to intimately conform to a skin surface without high resistance air gaps. Furthermore, conventional electronic constructions may create a low thermal resistance between thermal actuators and multiple temperature sensors, thereby limiting relative signal response to the object of interest. Additional challenges may also exist due to influence of undesirable external factors, such as air flow over the sensors, which may reduce the signal reliability of body tissues below the skin. In the case of thermal fluid sensors, a specific orientation of the sensor relative to the underlying fluid may provide improved signal levels. Some mechanical designs may optimize usability of the sensor for alignment to an underlying region of interest. Embodiments described herein serve to address one or more of these challenges by employing multiple constructions to optimize signals by modifying the mechanical designs. Furthermore, wearable thermal sensors require a combination of intimate thermal sensor contact and comfortable wearability from a combination of low mass and comfortable form factor. Some of the currently existing designs may be unable to achieve each parameter simultaneously due to either large mass or rigid and uncomfortable form factor. Embodiments disclosed herein describe constructions to resolve each issue simultaneously. In addition to the above-mentioned challenges, other challenges related to electrical signals may exist with skin-mountable thermal sensors. Thermal actuators applied to tissue, such as skin, may represent a hazard to the user if the thermal actuation creates excess heat. Conventional skin-mounted thermal actuators do not account for possible tissue variations in constant power systems, or possible component failure in constant temperature systems, or do not account for expected component variability in manufacturing. Embodiments disclosed herein describe constructions for creating hardware-controlled, software-controlled, and redundant thermal failsafe systems. Furthermore, conventional systems may be tethered to an external power source, such as wall power, instead of operating wirelessly or using a battery. This tethered setup introduces challenges, such as patient mobility, patient comfort, reduce visibility of insertion site, increase the risk of cord entanglement, and cluttering of the bedside area, which can lead to unintentional dislodgment of the IV catheter. Some embodiments described here integrate wireless power systems, and rechargeable batteries, eliminating tethers and enabling a conformal device that directly adheres to the skin for continuous, uninterrupted monitoring. Further, conventional skin-mounted thermal actuation sensors require large batteries, suffer short shelf-life, or require mechanical openings to charge or replace batteries, which may act as ingress points for damage, safety issues, or signal perturbations. Some embodiments disclosed herein enable solutions for compact, long shelf-life, and hermetically sealed designs.

As a non-limiting example, the monitoring of unintended fluid leakage (e.g., fluid infiltration or extravasation) beneath the skin during intravenous therapies and procedures represents an exemplary condition for noninvasive fluid measurement. Peripheral Intravenous Catheters (PIVCs) are essential components in healthcare, enabling the direct delivery of fluids, medications, and therapeutic agents into the bloodstream. PIVCs are utilized in over 80% of hospitalized patients, with more than 300 million devices sold annually in the U.S. alone. Despite their widespread use, complications such as infiltration and extravasation pose significant challenges. Infiltration is characterized by fluid leakage and buildup in the surrounding tissue resulting in pain, while extravasation can escalate to devastating outcomes such as compartment syndrome, nerve damage, scarring, and even the risk of amputation, particularly with highly reactive medications such as norepinephrine. Although patients of all ages are susceptible to PIVC complications (30-50%), vulnerable populations such as pediatrics and neonates face heightened risks due to their smaller vessel vasculature and limited ability to communicate pain. Unfortunately, preterm infants are exceptionally vulnerable, as extravasation can swiftly lead to loss of life due to their low subcutaneous fat. Incident rates of PIVC complications for vulnerable populations is reported up to 75% and up to 65% for infiltration and extravasation. Due to the rates of PIVC complications, hospital guidelines require frequent monitoring of vulnerable patients every 5 to 60 minutes and replacement every 96 hours. This, however, imposes strain on healthcare resources, demanding additional staff time and diminishing the focus of nursing staff. Complications often go unnoticed for extended periods, with studies revealing an average detection time of ˜45 hours by clinicians. These complications result in delays in medication delivery, dosing inaccuracies, and tissue damage, imposing a substantial economic burden on healthcare costs totaling up to 500 million dollars a year in the US. As a result, there exists a critical unmet clinical need for continuous monitoring for early detection of PIVC complications.

The day-to-day complexities faced when managing peripheral intravenous catheters include: (i) difficulties in frequent monitoring due to parental requests for “hands-off time”, for family privacy or to allow the child an uninterrupted sleep; (ii) challenges in monitoring when the patient is covered with wraps or surgical drapes to maintain a sterile environment, as breaking the barrier poses a risk of infection; (iii) issues related to bedside real estate in most rooms; and (iv) the attachment of cords to the skin, notably with pediatric patients, increasing the potential for IV dislodgment when the child is taken out of the crib. These challenges highlight the need for real-time monitoring for PIVC complications that integrate with PIVC work flows and available real estate in the hospital and on the skin.

While commercially available devices, such as ivWatch®, are designed to detect infiltration and extravasation, they face limitations in their ability to be widely adopted in hospitals. These systems are large pieces of equipment that are tethered to the patient. This poses issues with bedside real estate and raises concerns about additional cords close to the intravenous (IV) line, which risk dislodgment. In the case of ivWatch®, optical approaches prove ineffective in the presence of dark, colored, or cloudy fluids. Additionally, its functionality is compromised when covered or the skin is pigmented, potentially due to bruises or tattoos. Some existing devices use electrical impedance monitoring which is heavily impacted by the ionic concentration of the fluid and is designed to monitor bolus flow of ionic contrast agents. Both modalities are limited in their size, tethered design, and use case depending on IV fluid properties. In contrast, thermal imaging offers a cable-free solution, unaffected by issues related to fluid color or ionic concentration. However, thermal imaging systems require a direct line of sight and are susceptible to motion artifacts. Available systems face similar challenges such as high capital costs, scalability issues, and limited accessibility, thus limiting their adoption in all hospitals. Accordingly, there is a need for a precise, rapid, easy-to-use, wireless, non-invasive fluid sensor that can be used to monitor subdermal fluids including for infiltration and extravasation events associated with intravenous procedures. The proposed systems and devices provide a seamless, scalable, and cable-free solution for continuous monitoring of infiltration and extravasation wirelessly.

### SUMMARY

Embodiments of the present disclosure provide systems and methods of monitoring fluids beneath the skin surface on a body. Disclosed herein are exemplary systems and methods of measurements of fluids follow from spatial mapping of skin temperature over time and/or localized thermal actuation and sensing using a flexible, non-invasive device gently laminated onto the surface of the skin at a location of interest such nearby to an intravenous needle or catheter insertion site. In addition to fluid infiltration and extravasation, the system may also detect other physiological changes, such as phlebitis, tissue ischemia, infection, and edema, based on abnormal thermal patterns or temperature changes. The results presented here extend these concepts into a user-friendly, fully wireless system that enables continuous, noninvasive monitoring of fluid infiltration performed by clinicians or by patients themselves in real-world settings. Advanced designs and integration schemes exploit low-cost commercial components and flexible circuit board manufacturing techniques in optimized layouts guided by theoretical and numerical models of thermal transport and system-level mechanics. In some embodiments, a Bluetooth Low-Energy System on a Chip (BLE-SoC) embedded system architecture allows for robust, high-quality data transfer during normal patient activities, where a miniaturized on-board, rechargeable battery supports continuous operation over hours to days. Different embodiments of integration with adhesive materials support different application use cases to provide flexibility of use.

The closest references are Webb, R Chad et al. “Ultrathin conformal devices for precise and continuous thermal characterization of human skin.” Nature materials, vol. 12, 10 (2013): 938-44, “System and method for mitigating the effects of tissue blood volume changes to aid in diagnosing infiltration or extravasation in animalia tissue,” (U.S. Pat. No. 9,326,686 B2), and Matsui, Yuko et al. “Evaluation of the Predictive Validity of Thermography in Identifying Extravasation With Intravenous Chemotherapy Infusions.” Journal of Infusion Nursing: the official publication of the Infusion Nurses Society vol. 40, 6 (2017): 367-374. While each reference is associated with either thermal mapping on skin or detecting intravenous infiltration events, none of the references, either independently or in combination, teaches the features of using an array of temperature sensors, combined with an array of thermal actuators with actuator size large enough and actuation time long enough to create a thermal penetration depth of several millimeters or more into tissue. Such system then enables calculation of parameters based on two-dimensional distribution of temperatures over time to calculate a condition related to possible fluid infiltration. This specific combination of features provides benefit over the prior art by resulting in a system which can detect infiltration over a spatial area (unlike point source optical signals such as U.S. Pat. No. 9,326,686 B2), while also using changes in thermal transport in deep infiltrations which often do not result in reliable temperature change at the skin surface, which is a limitation of any system relying on only temperature changes (such as Webb and Matsui). Other scenarios where temperature mapping alone is limited include infusions of warmed fluid, or slower infusions where the fluid may equilibrate to body temperature. While Webb demonstrates the use of thermal actuation, the small size and short actuation time of the sensors do not support detection of fluids beyond 1 mm beneath surface as the system is designed for monitoring surface skin hydration, and other references using larger actuators for deeper thermal penetration do obviously translate to systems which enable spatial mapping of signals to identify intravenous infiltration events.

Some embodiments of the present disclosure are directed to a system for monitoring subdermal fluid infiltration. The system may include a flexible substrate configured to be adhered to a skin surface; at least six temperature sensors supported by the flexible substrate and arranged spatially in a first array; at least three thermal actuators supported by the flexible substrate and arranged spatially in a second array; an adhesive substrate supported by the flexible substrate; a power source configured to supply power to the at least six temperature sensors and to the at least three thermal actuators; a memory storage device; and a microprocessor in communication with the memory storage device. The microprocessor may be configured to activate the power source to supply power to at least one of the at least three thermal actuators to cause heating of a subdermal fluid; receive information associated with a temperature of the subdermal fluid from at least one of the at least six temperature sensors; calculate an alert condition based on a set of instructions received from a memory storage device and the temperature of the subdermal fluid; and trigger an alert if the alert condition is met.

The system may further include a graphical user interface (GUI) in communication with the microprocessor, and the GUI may be configured to display a spatial map of the temperature of the subdermal fluid. The first array may comprise a rectangular array of sixteen temperature sensors or twenty-five temperature sensors. Each of sixteen temperature sensors is configured to detect the temperature of the subdermal fluid within a region of at least 4 cm². Each of twenty-five temperature sensors is configured to detect the temperature of the subdermal fluid within a region of at least 4 cm².

The second array may include four thermal actuators or six thermal actuators. An area of each of the four or six thermal actuators is at least 30 mm². The power source may be configured to apply power to a thermal actuator to cause heating of the subdermal fluid for at least sixty seconds. The system may comprise twenty-five temperature sensors and six thermal actuators supported by the flexible substrate.

At least 30% of a portion of the flexible substrate comprising the at least six temperature sensors and the at least three thermal actuators may be optically transparent or substantially transparent. The spatial map comprises a two-dimensional temporal distribution of temperatures of the subdermal fluid. A calculation of the alert condition comprises a measurement of a curvature of an isotherm line in the spatial map of the temperature of the subdermal fluid.

The at least six temperature sensors and the at least three thermal actuators may be disposed on the adhesive substrate. The at least six temperature sensors and the at least three thermal actuators form a removable electrical connection with the power source, the microprocessor, and the memory storage device. The alert may comprise a notification of an occurrence of an infiltration event, and the notification comprises a visual notification, an audio notification, a haptic notification, or an audio-visual notification.

Some embodiments of the present disclosure are directed to a method for monitoring subdermal fluid infiltration using a device comprising a flexible substrate, at least six temperature sensors, at least three thermal actuators, a power source, a microprocessor, and a memory storage device. The method may include activating the power source to supply power to at least one of the at least three thermal actuators to cause heating of a subdermal fluid; receiving information associated with a temperature of the subdermal fluid from at least one of the at least six temperature sensors; calculating an alert condition based on a set of instructions received from the memory storage device and the temperature of the subdermal fluid; and triggering an alert if the alert condition is met.

The method may further include generating a spatial map of the temperature of the subdermal fluid; displaying the spatial map on a graphical user interface (GUI) associated with the device; measuring a curvature of an isotherm line in the spatial map; and determining an occurrence of an infiltration of the subdermal fluid based on the spatial map.

### BRIEF DESCRIPTION OF THE DRAWINGS

FIGS. 1A and 1B are illustrations of exemplary applications of thermal fluid sensors associated with intravenous catheter placements, consistent with some disclosed embodiments.

FIG. 2A illustrate an exemplary arrangement of electrical components of a thermal fluid sensor consolidated within one physical system, consistent with some disclosed embodiments.

FIGS. 2B and 2C illustrate an exemplary arrangement of electrical components of a thermal fluid sensor where the sensor/actuator components are mechanically separable from other components, consistent with some disclosed embodiments.

FIGS. 3A and 3B illustrate an exemplary arrangement of sensors and/or actuators in a thermal fluid flow sensor, consistent with some disclosed embodiments.

FIG. 4 illustrate an exemplary arrangement of layers of a thermal fluid flow sensor, consistent with some disclosed embodiments.

FIG. 5 illustrate an exemplary patterning of a thermal fluid sensor, consistent with some disclosed embodiments.

FIG. 6 illustrates an exemplary arrangement of layers of a thermal fluid flow sensor, consistent with some disclosed embodiments.

FIG. 7 illustrates an arrangement of components of a thermal fluid sensor supporting strap-mounting portion on a limb, consistent with some disclosed embodiments.

FIG. 8 illustrates an exemplary arrangement of layers of a thermal fluid flow sensor, consistent with some disclosed embodiments.

FIG. 9 illustrates an exemplary arrangement of layers of a thermal fluid flow sensor, consistent with some disclosed embodiments.

FIG. 10 illustrates an exemplary arrangement of layers of a thermal fluid flow sensor, consistent with some disclosed embodiments.

FIGS. 11A and 11B illustrate an exemplary spatial patterning of a thermal fluid flow sensor and conductive traces, consistent with some disclosed embodiments.

FIG. 12 illustrates an exemplary spatial patterning of a thermal fluid flow sensor, consistent with some disclosed embodiments.

FIGS. 13A and 13B illustrate exemplary arrangements of thermal actuators and sensors of a thermal fluid flow sensor, consistent with some disclosed embodiments.

FIGS. 14A and 14B illustrate exemplary patterning a thermal fluid flow sensor to support stretchability and flexibility, consistent with some disclosed embodiments.

FIG. 15 illustrates an exemplary spatial patterning of a thermal fluid flow sensor, consistent with some disclosed embodiments.

FIGS. 16A and 16B illustrate examples of temperature data monitoring from a thermal fluid sensor associated with no infiltration event and an infiltration event, respectively, consistent with some disclosed embodiments.

FIGS. 17A-C and 17D-F illustrate examples of temperature data monitoring from a thermal fluid sensor associated with no infiltration event and an infiltration event, respectively, consistent with some disclosed embodiments.

FIGS. 18A and 18B illustrate examples of temperature data monitoring associated with thermal actuator use from a thermal fluid sensor associated with no infiltration event and an infiltration event, consistent with some disclosed embodiments.

FIGS. 19A-C illustrate an example of temperature data monitoring from a thermal fluid sensor using a thermal actuator array associated with an infiltration event, consistent with some disclosed embodiments.

FIG. 20 illustrates a system combining a thermal fluid sensor with a thermal actuation of an intravenous fluid, consistent with some disclosed embodiments.

FIG. 21A illustrates an exemplary thermal fluid sensor, consistent with some disclosed embodiments.

FIG. 21B illustrates an exemplary thermal fluid sensor which alerts a user through an additional electronic device, consistent with some disclosed embodiments.

FIG. 22 illustrates an exemplary arrangement of multiple thermal fluid sensors wirelessly connected to a central processor which alerts a user through a graphical user interface (GUI), consistent with some disclosed embodiments.

FIG. 23 illustrates a thermal actuator safety circuit and a temperature sensor amplifier signal circuit, consistent with some disclosed embodiments.

FIG. 24A illustrates an exemplary arrangement of thermal sensors and thermal actuators, consistent with some disclosed embodiments.

FIG. 24B illustrates an exemplary skin-mountable monitoring device, consistent with some disclosed embodiments.

FIG. 25 illustrates an exemplary profile of temperature data monitoring of a thermal fluid sensor of FIG. 24A, consistent with some disclosed embodiments.

FIG. 26 illustrates two-dimensional spatial thermal maps showing heat distribution in response to thermal actuation, consistent with some disclosed embodiments.

FIG. 27 illustrates a comparison of the monitoring and detection of infiltration events based on the dimensions of the thermal actuators, consistent with some disclosed embodiments.

### DETAILED DESCRIPTION

Reference will now be made in detail to exemplary embodiments, examples of which are illustrated in the accompanying drawings. The following description refers to the accompanying drawings in which the same numbers in different drawings represent the same or similar elements unless otherwise represented. The implementations set forth in the following disclosure. Instead, they are merely examples of apparatuses and methods consistent with aspects related to the subject matter recited in the appended claims. The following detailed description refers to the accompanying drawings. While several illustrative embodiments are described herein, modifications, adaptations and other implementations are possible. For example, substitutions, additions, or modifications may be made to the components and steps illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope of the invention is defined by the appended claims.

Relative dimensions of components in drawings may be exaggerated for clarity. Within the following description of drawings, the same or like reference numbers refer to the same or like components or entities, and only the differences with respect to the individual embodiments are described.

As used herein, unless specifically stated otherwise, the term “or” encompasses all possible combinations, except where infeasible. For example, if it is stated that a database may include A or B, then, unless specifically stated otherwise or infeasible, the database may include A, or B, or A and B. As a second example, if it is stated that a database may include A, B, or C, then, unless specifically stated otherwise or infeasible, the database may include A, or B, or C, or A and B, or A and C, or B and C, or A and B and C.

In general, the terms and phrases used herein have their art-recognized meaning, which can be found by reference to standard texts, journal references and contexts known to those skilled in the art. The following definitions are provided to clarify their specific use in the context of the invention.

In this context, “soft” refers to a material that may be comfortably positioned against the skin without discomfort or irritation to the underlying skin by the material itself deforming to conform to the skin without unduly exerting force on the underlying skin with corresponding device-generated skin deformation. Softness/hardness may be optionally quantified, such as in terms of durometer, or a material's resistance to deformation. For example, the substrate may be characterized in terms of a Shore 00 hardness scale, such as a Shore 00 that is less than 80. Soft may also be characterized in terms of a modulus, such as a Young's modulus that is less than or equal to 100 kPa.

In this context, “stretchable” refers to a material's ability to undergo reversible deformation under an applied strain. This may be characterized by a Young's modulus, a ratio of stress to strain. A bulk or effective Young's modulus refers to a composite material formed from materials having different Young's modulus, so that the bulk or effective Young's modulus is influenced by each of the different materials and provides an overall device-level modulus.

In this context, “flexible” refers to a material's ability to undergo a bending without fracture or permanent deformation, and may be described in terms of a bending modulus.

Any of the devices may be described herein as being “mechanically matched” to skin, specifically the skin over which the device will rest. This matching of device to skin refers to a conformable interface, for example, useful for establishing conformal contact with the surface of the tissue. Devices and methods may incorporate mechanically functional substrates comprising soft materials, for example exhibiting flexibility and/or stretchability, such as polymeric and/or elastomeric materials. A mechanically matched substrate may have a modulus less than or equal to 100 MPa, less than or equal to 10 MPa, or less than or equal to 1 MPa. A mechanically matched substrate may have a thickness less than or equal to 0.5 mm, and optionally for some embodiments, less than or equal to 1 cm, and optionally for some embodiments, less than or equal to 3 mm. A mechanically matched substrate may have a bending stiffness less than or equal to 1 N/m, optionally less than or equal to 0.1 N/m.

A mechanically matched device, and more particularly a substrate is characterized by one or more mechanical properties and/or physical properties that are within a specified factor of the same parameter for an epidermal layer of the skin, such as a factor of 10 or a factor of 2. For example, a substrate may have a Young's Modulus or thickness that is within a factor of 20, or optionally for some applications within a factor of 10, or optionally for some applications within a factor of 2, of a tissue, such as an epidermal layer of the skin, at the interface with a device of the present invention. A mechanically matched substrate may have a mass or modulus that is equal to or lower than that of skin.

In this context, “encapsulate” refers to the orientation of one structure such that it is at least partially, and in some cases completely, surrounded by one or more other structures, such as a substrate, adhesive layer, or encapsulating layer. In this context, “partially encapsulated” refers to the orientation of one structure such that it is partially surrounded by one or more other structures, for example, wherein 30%, or optionally 50%, or optionally 90% of the external surface of the structure is surrounded by one or more structures. In this context, “completely encapsulated” refers to the orientation of one structure such that it is completely surrounded by one or more other structures.

In this context, “polymer” refers to a macromolecule composed of repeating structural units connected by covalent chemical bonds or the polymerization product of one or more monomers, often characterized by a high molecular weight. The term polymer includes homopolymers, or polymers consisting essentially of a single repeating monomer subunit. The term polymer also includes copolymers, or polymers consisting essentially of two or more monomer subunits, such as random, block, alternating, segmented, grafted, tapered and other copolymers. Useful polymers include organic polymers or inorganic polymers that may be in amorphous, semi-amorphous, crystalline or partially crystalline states. Crosslinked polymers having linked monomer chains are particularly useful for some applications. Polymers useable in the methods, devices and components disclosed include, but are not limited to, plastics, elastomers, thermoplastic elastomers, elasto-plastics, thermoplastics and acrylates. Exemplary polymers include, but are not limited to, acetal polymers, biodegradable polymers, cellulosic polymers, fluoropolymers, nylons, polyacrylonitrile polymers, polyamide-imide polymers, polyimides, polyacrylates, polybenzimidazole, polybutylene, polycarbonate, polyesters, polyetherimide, polyethylene, polyethylene copolymers and modified polyethylenes, polyketones, poly(methyl methacrylate), polymethylpentene, polyphenylene oxides and polyphenylene sulfides, polyphthalamide, polypropylene, polyurethanes, styrenic resins, sulfone-based resins, vinyl-based resins, rubber (including natural rubber, styrene-butadiene, polybutadiene, neoprene, ethylene-propylene, butyl, nitrile, silicones), acrylic, nylon, polycarbonate, polyester, polyethylene, polypropylene, polystyrene, polyvinyl chloride, polyolefin or any combinations of these.

In this context, “elastomer” refers to a polymeric material which can be stretched or deformed and returned to its original shape without substantial permanent deformation. Elastomers commonly undergo substantially elastic deformations. Useful elastomers include those comprising polymers, copolymers, composite materials or mixtures of polymers and copolymers.

Elastomeric layer refers to a layer comprising at least one elastomer. Elastomeric layers may also include dopants and other non-elastomeric materials. Useful elastomers include, but are not limited to, thermoplastic elastomers, styrenic materials, olefinic materials, polyolefin, polyurethane thermoplastic elastomers, polyamides, synthetic rubbers, PDMS, polybutadiene, polyisobutylene, poly(styrene-butadiene-styrene), polyurethanes, polychloroprene and silicones. Exemplary elastomers include, but are not limited to silicon containing polymers such as polysiloxanes including poly(dimethyl siloxane) (i.e. PDMS and h-PDMS), poly(methylsiloxane), partially alkylated poly(methyl siloxane), poly(alkyl methyl siloxane) and poly(phenyl methyl siloxane), silicon modified elastomers, thermoplastic elastomers, styrenic materials, olefinic materials, polyolefin, polyurethane thermoplastic elastomers, polyamides, synthetic rubbers, polyisobutylene, poly(styrene-butadiene-styrene), polyurethanes, polychloroprene and silicones. In an embodiment, a polymer is an elastomer.

In this context, “conformable” refers to a device, material or substrate which has a bending stiffness that is sufficiently low to allow the device, material or substrate to adopt any desired contour profile, for example a contour profile allowing for conformal contact with a surface having a pattern of relief features. In some embodiments, a desired contour profile is that of a human skin.

In this context, “conformal contact” refers to contact established between a device and a receiving surface, specifically skin. In one aspect, conformal contact involves a macroscopic adaptation of one or more surfaces (e.g., contact surfaces) of a device to the overall shape of a surface. In another aspect, conformal contact involves a microscopic adaptation of one or more surfaces (e.g., contact surfaces) of a device to a surface resulting in an intimate contact substantially free of voids. In some embodiments, conformal contact may involve adaptation of a contact surface(s) of the device to a receiving surface(s) such that intimate contact is achieved, for example, wherein less than 20% of the surface area of a contact surface of the device does not physically contact the receiving surface, or optionally less than 10% of a contact surface of the device does not physically contact the receiving surface, or optionally less than 5% of a contact surface of the device does not physically contact the receiving surface. Devices of certain aspects are capable of establishing conformal contact with internal and external tissue. Devices of certain aspects are capable of establishing conformal contact with tissue surfaces characterized by a range of surface morphologies including planar, curved, contoured, macro-featured and micro-featured surfaces and any combination of these. Devices of certain aspects are capable of establishing conformal contact with tissue surfaces corresponding to tissue undergoing movement.

In this context, “Young's modulus” is a mechanical property of a material, device or layer which refers to the ratio of stress to strain for a given substance. “Low modulus” refers to materials having a Young's modulus less than or equal to 10 MPa, less than or equal to 5 MPa or less than or equal to 1 MPa.

In this context, “bending stiffness” is a mechanical property of a material, device or layer describing the resistance of the material, device or layer to an applied bending moment. Bending stiffness is defined as the product of the modulus and area moment of inertia of the material, device or layer. A material having an inhomogeneous bending stiffness may optionally be described in terms of a “bulk” or “average” bending stiffness for the entire layer of material.

Here, “Bluetooth Low-Energy System on a Chip (BLE-SOC)” is a specialized electronic component designed to enable wireless communication between a device and external systems, such as computers or smartphones. BLE-SoC is built on Bluetooth Low Energy technology, which is optimized for low power consumption, making it suitable for battery-operated devices like medical wearables. This component is fully compatible with modern devices and incorporates advanced encryption and data protection protocols, ensuring secure transmission of sensitive patient information in compliance with healthcare standards.

In this context, “thermal dynamics” refers to the broad understanding of how heat transfers through a material or across surfaces. These properties are critical for understanding the behavior of thermal actuators and sensors in devices that monitor physiological parameters. The primary thermal properties we will include are: (a) “thermal conductivity” referred to as the ability of a material to conduct heat, determining how effectively heat is transferred within the material; (b) “heat capacity” referred to as the material's ability to store thermal energy; (c) “thermal diffusivity” referred to as the rate at which heat spreads through a material, dependent on both thermal conductivity and heat capacity; (d) “effusivity” referred to as a measure of how easily a material exchanges heat with its environment, important in thermal sensing; and (f) “convection” referred to as the transfer of heat by the movement of fluids or gas, important when dealing with biological tissues and wearable devices.

In this context, “PIVC complications” refers to the range of common complications that may be encountered during PIVC therapy. These include, but are not limited to, conditions such as “infiltration” where IV fluids leak into surrounding tissues; “extravasation” where the IV fluid materials contain harmful medications that can leak into surrounding tissues causing potential tissue damage, “Phlebitis” referring to the inflammation of the veins due to catheter placement, or “Ischemia” which occurs when blood flow to tissues is reduced, often causing localized cooling. The device is designed to monitor these complications through temperature changes and other physiological signals, enabling early detection and timely intervention to improve patient outcomes.

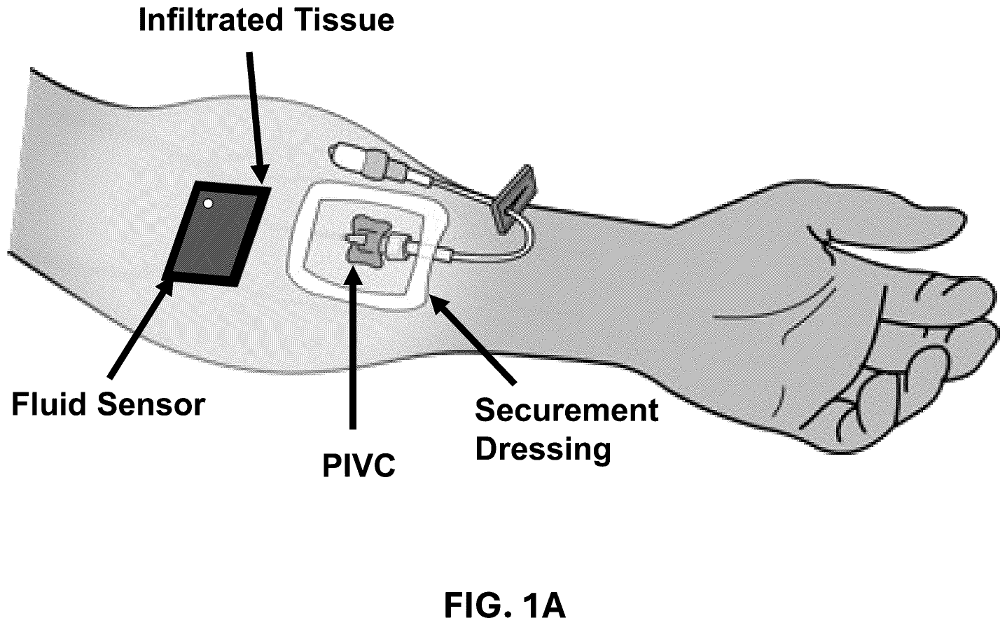

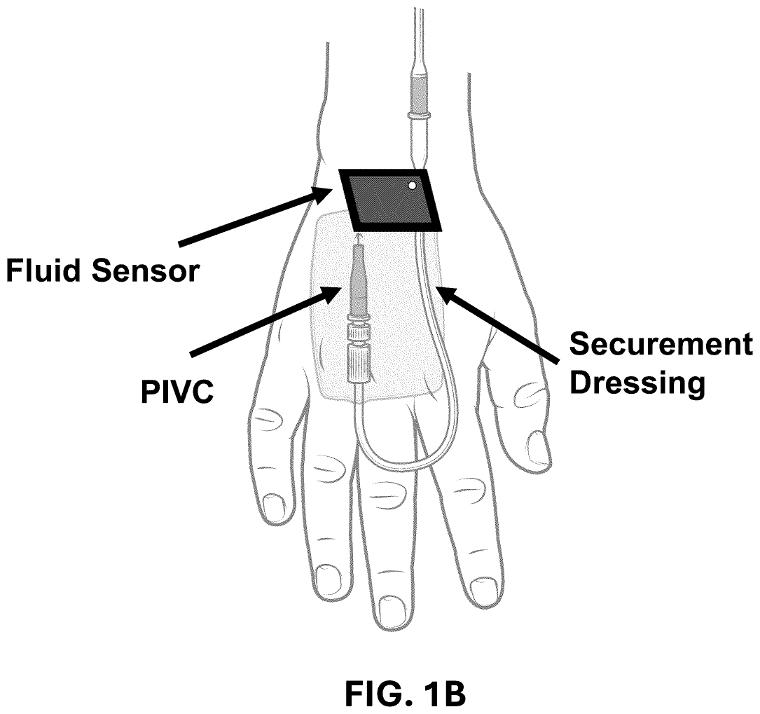

Reference is now made to FIG. 1, which illustrates an anatomy of a typical PIVC insertion point in the upper arm and an exemplary application of a wireless fluid sensor to the region. A wireless, flexible, non-invasive fluid sensor may be placed on a skin surface in the proximity of the PIVC to detect an IV infiltration event. In some embodiments, the fluid sensor may be an integrated patch adhered to a skin surface via an adhesive at a position either proximal or distal to the PIVC insertion point as appropriate. In such an embodiment, the sensor, actuator, control, communication and power electronics may be integrated into a single mechanical element which is applied to the skin. Multiple uses of such an embodiment may be facilitated by a surface which supports a replaceable adhesive patch, such that an adhesive may be removed for disposal after use, and a new adhesive may be applied for further use. In other embodiments, the fluid sensor region may be mechanically separable from power, control and communication electronics. Such an embodiment may facilitate integration of the direct sensing components, which may be primarily comprised of relatively low-cost components such as surface mount technology (SMT) thermistors and resistors wired with thin film conductive traces, directly into disposable adhesive materials. Such a sensor-adhesive patch integration may be connected to a physically separate module which contains other electronics, such as power, control and communication components, by a wired or wireless connection. In this way, the low-cost portion of the sensing components may be disposed of after each use, while the higher cost power, control and communication electronics may be reused across many sensor-adhesive patches. In some embodiments, the direct sensing components may be integrated directly into IV securement adhesive patches. In other examples, the direct sensing components may be integrated into a separate adhesive patch which may be applied to skin separate from or directly on top of IV securement patches.

Such a wireless thermal fluid sensor may identify fluid infiltration or extravasation events by monitoring thermal properties of tissue in regions local to the IV insertion site. The ingress of fluids into the surrounding tissue can result in a change in temperature and/or thermal transport properties (e.g. thermal conductivity, diffusivity and the like) of the overall body material due to the differing temperature and thermal transport properties of the IV fluid and/or blood relative to the other tissue such as skin, fat and muscle layers. In many IV infusion therapies the IV fluid is significantly colder, such as room temperature, than the body tissues and ingress of the IV fluid into tissues outside of the vein may be readily identified by temperature measurements of the skin surface alone, without need for additional information about thermal transport properties, or other non-thermal properties such as electrical impendence or optical absorption properties. In some embodiments, the addition of these further sensing modes, such as thermal transport measurements, may be provide additional data to improve the reliability of identifying IV infusion events such as infiltration and extravasation. For example, in some instances IV infusion fluids may be warmed to body temperature prior to infusion, such that fluid ingress into surrounding tissues may not be readily identifiable by temperature alone. In other instances, blood may pool into surrounding tissues which also may be difficult to identify by temperature alone. Temperature measurements may also be obfuscated by loss of adhesion of a portion of the sensor from the skin, which may cause sensors to identify lower temperatures that may appear similar to the ingress of IV fluid into tissues. In such instances, and other instances, the additional measurements of thermal transport properties may improve the identification of infiltration events. In some embodiments, one or more thermal actuators may be activated, via the application of additional power for a finite period of time such that the actuators increase in temperature. The rate and magnitude of such a temperature increase are functions of the thermal properties of the materials in thermal contact with the actuator, such that when the actuator or actuators are in contact with skin, their rate and magnitude of temperature increase are functions of the thermal properties of the surrounding tissues. An increase in diffusivity of the surround tissue, which may occur as a result of fluid accumulating in the tissue due to IV infiltration or extravasation, will typically result in a relative decrease in the temperature rise of the actuator for a given amount of power applied, such as between 0.1 mW/mm² and 10 mW/mm² and preferably between 1 mW/mm² and 5 mW/mm², wherein such applications of power may result in measurable increases in temperature without causing damage to skin. The rate at which the temperature of the actuator approaches an equilibrium temperature also changes, with the rate typically increasing for a locally increasing diffusivity. Such a system will also readily show opposite behavior in the case of delamination of the actuators from skin, which results in a decrease in the diffusivity in contact with the actuators and relatively larger magnitude of temperature increase. Thermal diffusivity α is known to related to other thermal properties by the relation:

$$α = \frac{k}{ρ {c}_{p}}$$

Where k is thermal conductivity (W/(m·K)), cₚ is specific heat capacity (J/(kg·K)), p is density (kg/m³). Thermal diffusivity is related to changes in temperature over time by:

$$\frac{δ T}{δ t} = α {∇}^{2} T$$

where Tis temperature and t is time. In practice, the heating of a transient plane source such as by thermal actuators described here may approximately follow the relationship:

$$Δ T ( τ ) = \frac{{P}_{0}}{4 a {π}^{1 / 2} k} H ( τ )$$

Where P₀ is the power output from the actuator element and approximated as constant during heating due to the <2% resistance change during heating, 2a is the width of the heater square, k is the thermal conductivity, ΔT is the average temperature rise in the heater, and r is given by

$$τ = {( \frac{α t}{{a}^{2}} )}^{1 / 2}$$

Condensed parameters for thermal transport properties may be calculated in many ways, such as by using known parameters such as applied power, area, and measured parameters such as temperature increase to calculate values such as thermal conductivity or diffusivity. In some embodiments, relative parameters may be used, such as the temperature increase of actuator 10 seconds, or 30 seconds, or 60 seconds or 300 seconds after applying power to the actuator relative to the baseline temperature before power was applied. In some embodiments, the temperature increase of the actuator may be measured by a separate temperature measurement component, such as a SMT thermistor, disposed locally to the thermal actuator element, which may be one or more SMT resistors. In some embodiments, the thermal actuator may be comprised of a conductive trace, and temperature monitoring may be accomplished by monitoring changes in resistance of the conductive trace, which may change resistivity as a function of temperature due to the temperature coefficient of resistance of the trace material or may other be monitoring by SMT thermistors disposed local to the conductive trace.

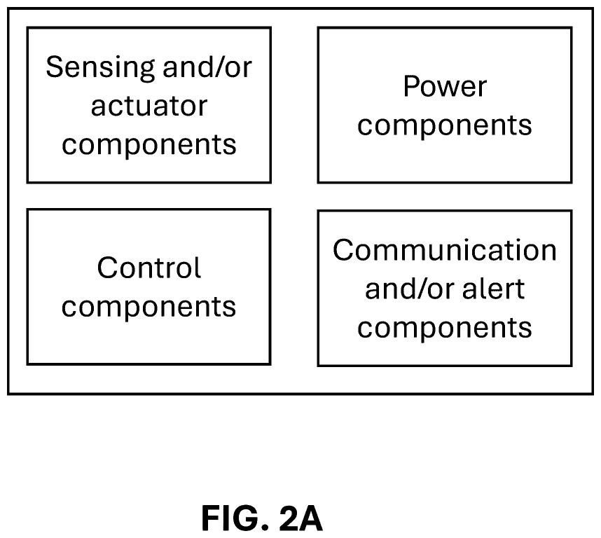

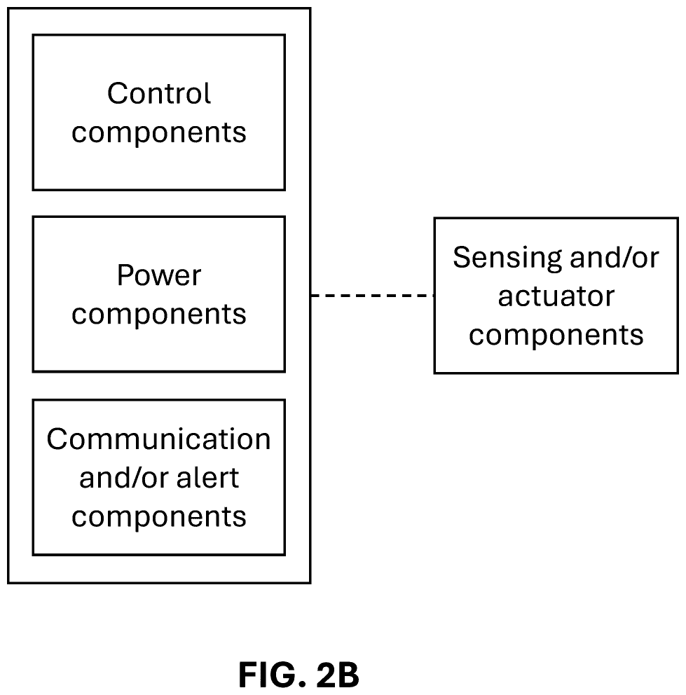

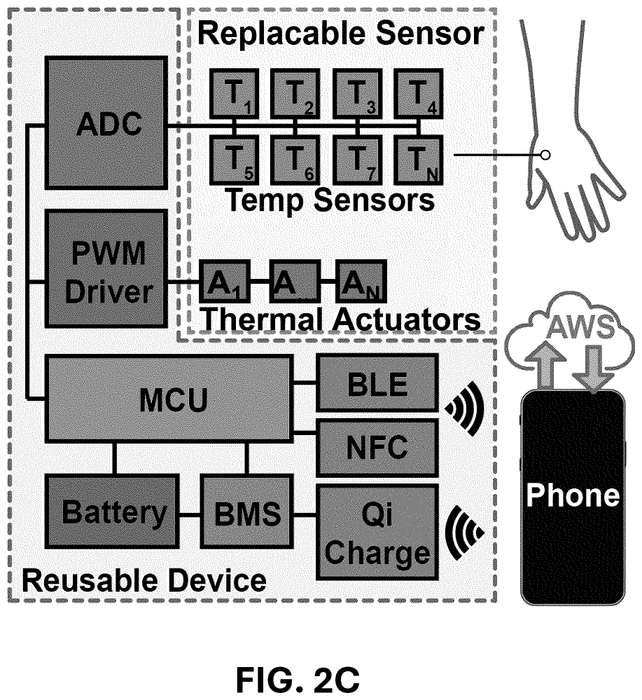

Reference is now made to FIG. 2A-C, which illustrates block diagrams of the components of the fluid sensor system, consistent with disclosed embodiments. In an exemplary embodiment illustrated by FIG. 2A all of the electronics components are integrated into a single physical unit. In some embodiments, as illustrated by FIG. 2B, the sensor/actuator unit is physically separable from the other electronic components such that the sensor/actuator component can be removed, such as plugging in or out, from the other electronics components. In some embodiments, as illustrated by FIG. 2C, the exemplary thermal fluid sensor comprises a reusable device containing a variety of electronic components and replaceable sensor unit containing additional electronic components. One or more of these components may be supported by the fPCB such that the components may be interconnected, if so desired. The components of the reusable device may include, but are not limited to, analog front-end circuitry to convert resistance measurements of temperature into corresponding output voltages, a microcontroller unit (MCU) and accompanying BLE-SoC (Bluetooth Low Energy System on Chip) and its associated timers and antenna to digitize and transmit these data, and also to support wireless two-way communication, power management electronics to supply power to the various sub-systems. The power management electronics may include a rechargeable lithium polymer (Li-Po) battery, a power management circuit, and an inductive coil for wireless power delivery through a protocol (e.g., Qi). Alternatively, the power management electronics may include a non-rechargeable primary battery, which may be replaceable by the end user. The components of the replaceable sensor may include temperature sensing and thermal actuating components such as SMT components. In this construction the replaceable sensor is comprised of relatively low-cost components supporting its use as a disposable unit which may be selectively connected such as via an FPC connector to the reusable device which is housed in a separate independent module. The fully wireless design of the proposed thermal fluid sensor represents a key advancement over existing technologies by allowing the patient to move freely while generating a continuous stream of data transmitted directly to a hand-held electronic device including, but not limited to, a smartphone, a tablet, a computer, a laptop, or any suitable electronic device having a graphical user interface (GUI) for displaying the data. This electronic device may be configurable to receive, transmit, store, upload, download, display data and further communicate with a wired or a wireless network such as Amazon Web Services (AWS).

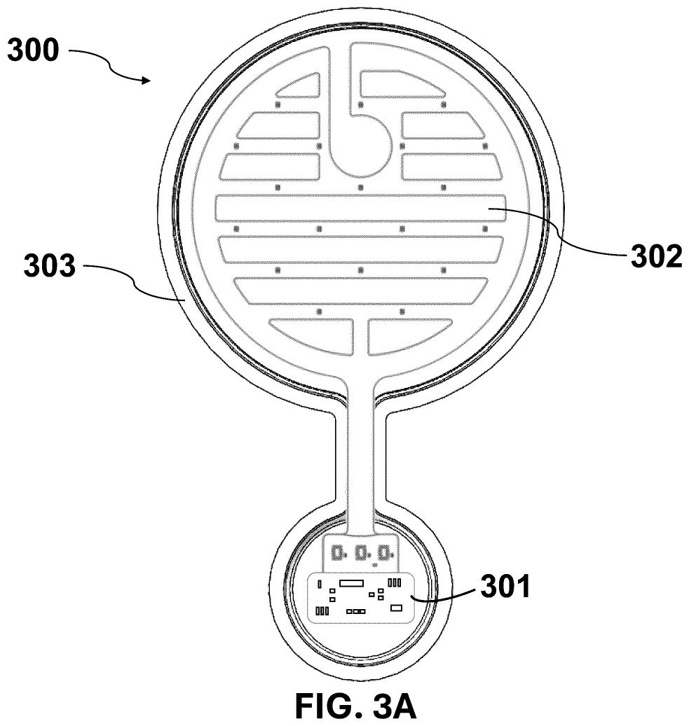

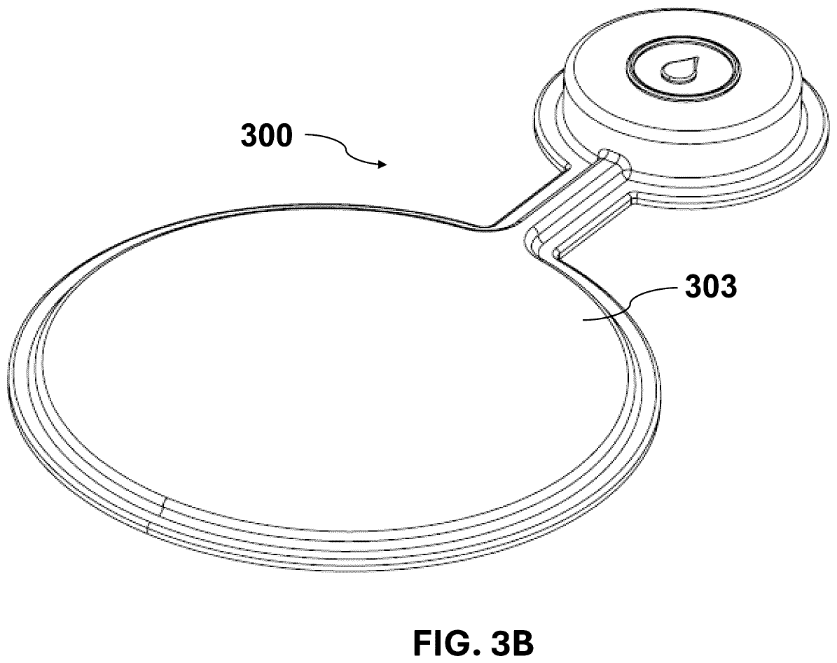

Reference is now made to FIG. 3A, which illustrates an exemplary structure of a wireless fluid sensor device, such as a wireless thermal fluid sensor 300. In some embodiments, the thermal fluid sensor 300 may comprise one or more electronics and one or more supporting layers 301 featuring patterned cutouts 302 and an enclosure structure 303. FIG. 3B illustrates a perspective view of a wireless thermal fluid sensor 300 showing enclosure structure 303.

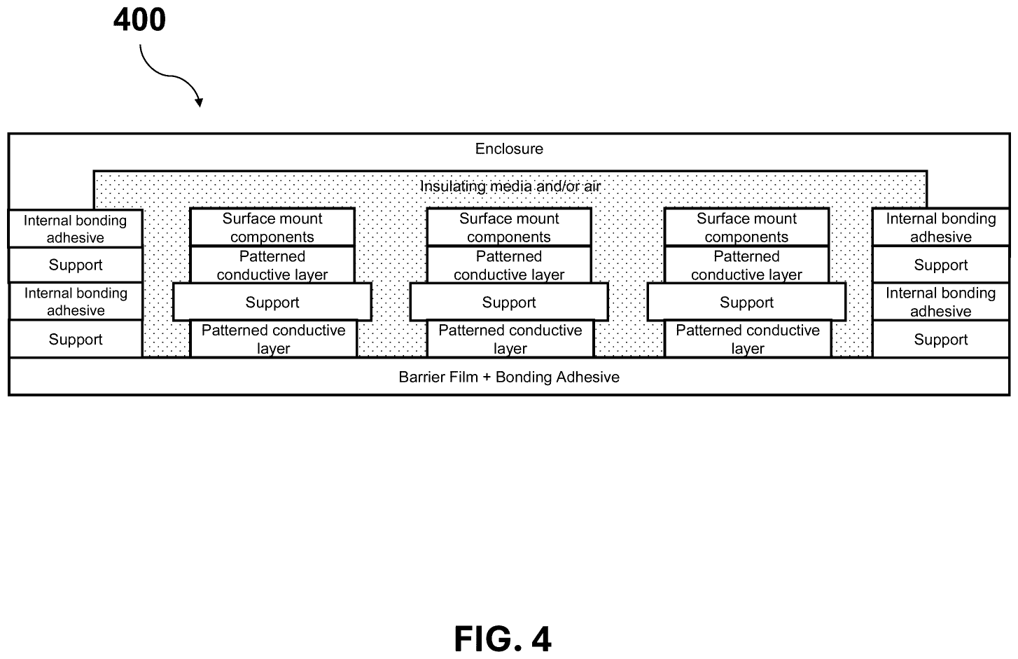

Referring now to FIG. 4, a thermal fluid sensor, also referred to herein as a fluid sensor 400, may comprise a variety of layers and geometrical arrangements conducive to thermal sensing or to thermal fluid sensing of infiltrated subdermal tissue. In some embodiments, fluid sensor 400 may comprise surface mount electrical components, one or more patterned conductive layers, a supporting layer such as, but not limited to, Kapton®, an insulating media such as foam or air, an enclosure, and a barrier layer such as a barrier film. In one exemplary construction, as part of the multilayer construction of the device, a barrier film may be applied to outermost Kapton® layer of the device to form the outermost layer of the device substrate. The barrier film may be comprised of a bonding adhesive on one side and a plastic film such as polyester on the other side. The bonding adhesive may preferably have an adhesion strength to the device such as 16 N/25 mm to provide an effectively permanent bond to the device. The plastic film backing may preferably have a thickness less than 0.1 mm and preferably less than 0.05 mm such as 0.03 mm. Such a construction may enable a high degree of mechanical flexibility and minimal thermal resistance through the barrier film. The barrier film layer may be used to protect the device from ingress by sealing the internal device components from the outside environment. The plastic polyester side of the barrier film may also feature a suitable surface finish or release coating be to promote the selective application and removal of an adhesive patch to and from the device. Various layers in a thermal fluid sensor may be patterned to create a variety of shapes and structures conducive to thermal measurements and product use. In some embodiments layers may be repeated, omitted, or replaced with other layers. For example, in yet another embodiment, one or more regions of a thermal fluid sensor may comprise rigid composite supporting layers, such as FR-4 commonly used in printed circuit board manufacturing and may include a laminate of multiple patterned conductive layers, such as 4 or 6 distinct patterned conductive layers, with surface mount components disposed on the top and bottom layers. Although not illustrated, it should be appreciated by an ordinarily skilled person in the art that a thermal fluid sensor may further comprise additional components within the sensor enclosure useful to wireless thermal fluid sensing, such as a power components, various sensing and actuating components, processing components, control components, wireless communication components, illuminating components, haptic components, or the like.

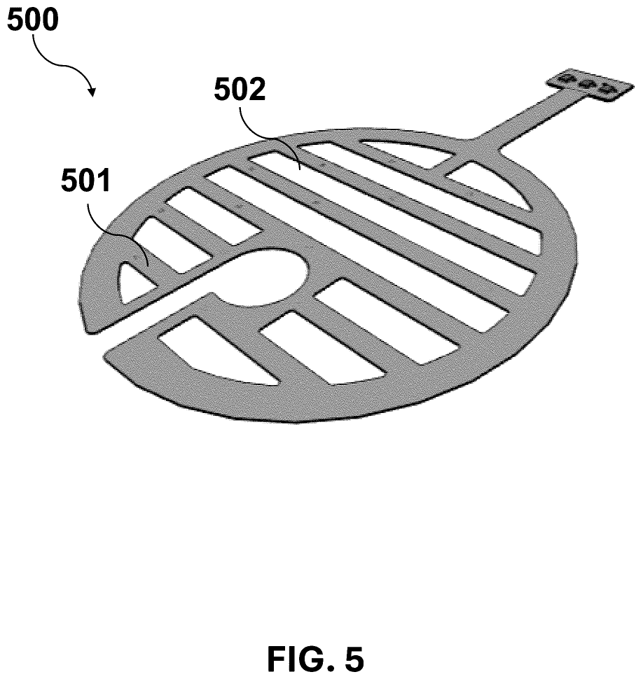

In some embodiments, it may be useful for the wireless thermal fluid sensor to be integrated into the existing dressing that is applied to the PIVC insertion site while still enabling visual inspection of the skin surface of the site. Reference is now made to FIG. 5, which illustrates an exemplary wireless fluid sensor 500, which may comprise one or more electronics and one or more supporting layers 501 featuring patterned cutouts 502.

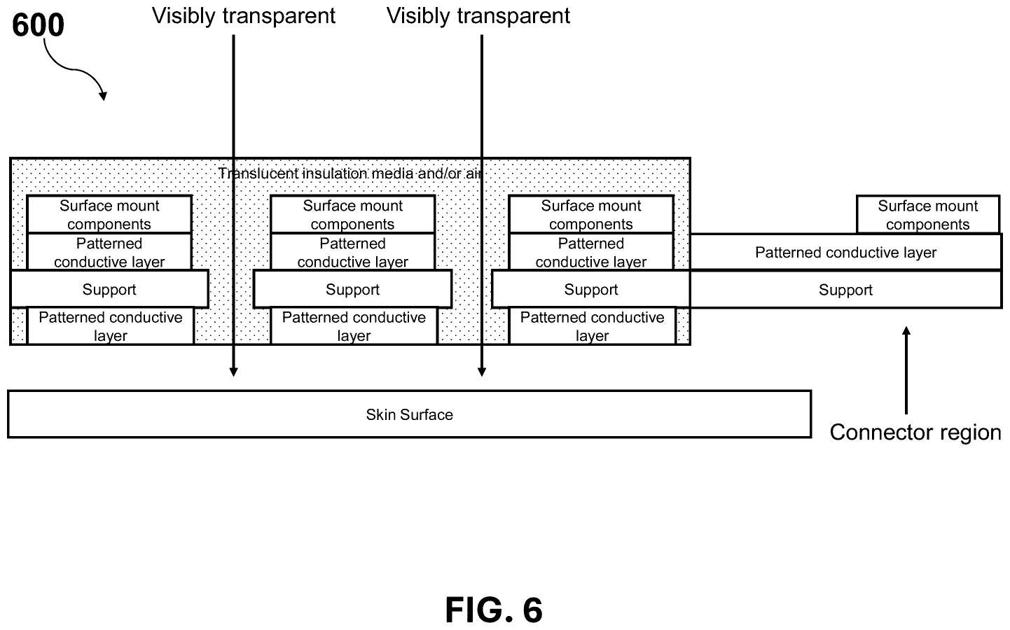

Referring now to FIG. 6, which illustrates a cross-section view of an exemplary thermal fluid sensor 600 comprising a plurality of layers and geometrical arrangements conducive to thermal sensing as previously disclosed. In some embodiments, a translucent (or a transparent) insulating media such as an aerogel or air may be used to insulate one or more of the SMT components. The use of a translucent media may enable visual inspection of the underlaying skin surface through the patterned cutouts of the supporting layers. Preserving the ability to visually observe this skin surface is useful as it complements existing diagnostic practices of visually monitoring the PIVC site for changes caused by fluid leakage such as discoloration or inflammation. In an exemplary construction the sensor 600 may also comprise an extended section featuring a flexible printed circuit (FPC) connector such as a zero-insertion force (ZIF), low insertion force (LIF), flip-lock, pogo-pin, or other connector. This connector may be used to connect to additional components useful to thermal fluid sensing such as power components, various sensing and actuating components, processing components, control components, wireless communication components, illuminating components, haptic components and the like which are housed in a region away from the PIVC insertion site. Additional sensing components include, but are not limited to, electrical impedance, mechanical impedance, optical absorption, ultrasound or acoustic.

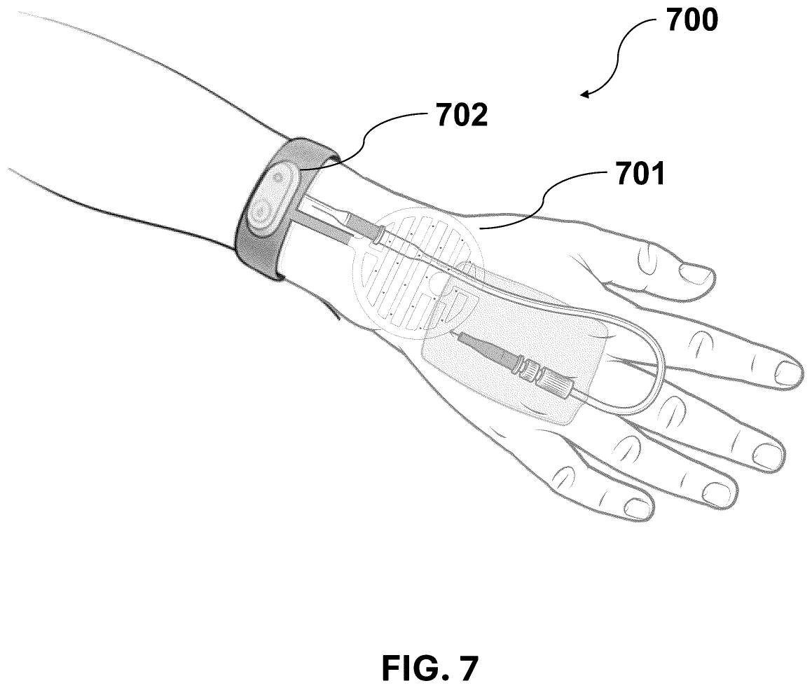

Referring to FIG. 7, a thermal fluid sensing system 700 may include a thermal fluid sensor 701 applied to the PIVC site which may be connected and disconnected from an additional module 702 such as a wrist worn strap housing additional power, communication, and sensing components. In some embodiments, the removeable thermal fluid sensor may be disconnected and disposed of when desired, such as when the IV dressing is to be replaced. In this construction, the thermal fluid sensor may be utilized as a low-cost disposable unit and a new thermal fluid sensor may be applied to the site and connected to the original wrist worn strap for continued monitoring. In some embodiments, the strap may be worn in other locations based on the location of the PIVC insertion site such as the arm, leg, or head. In further embodiments, the thermal fluid sensor may connect to a wearable patch that is adhered to the skin via an adhesive, rather than a compressive strap based on a user's comfort preferences.

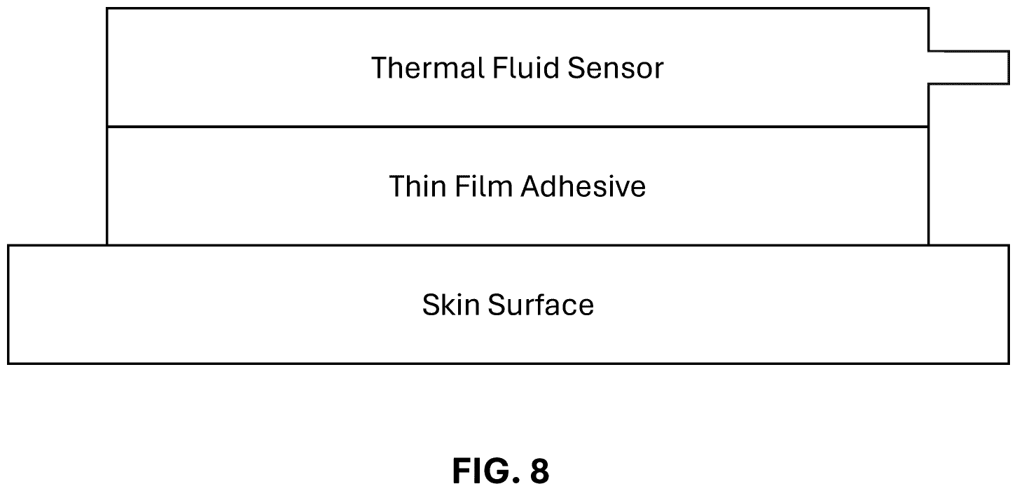

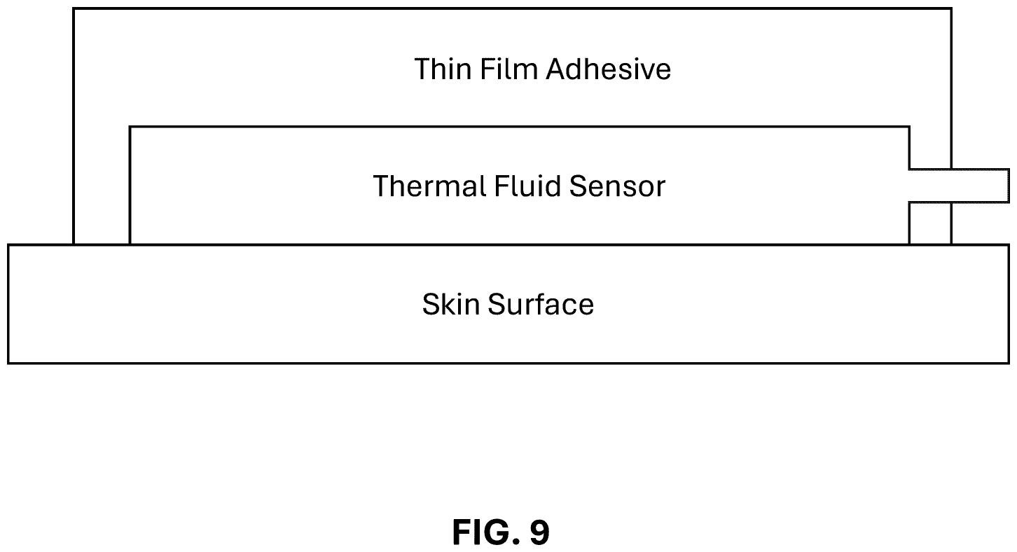

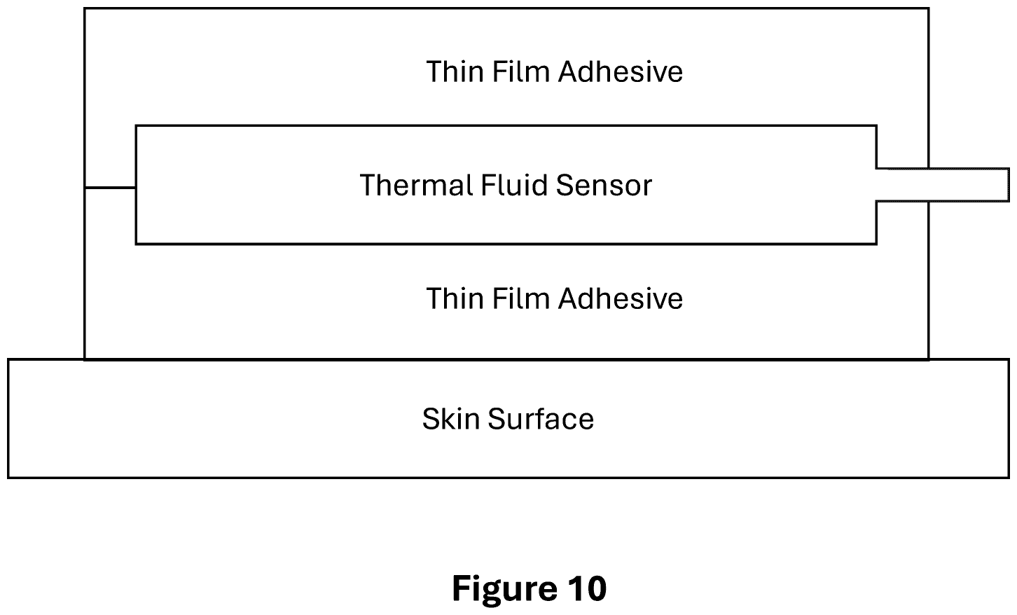

Referring to FIG. 8, an exemplary thermal fluid sensor 810 may be adhered to and thermally coupled with a skin surface 830 via a thin film adhesive 820 such as those used for IV securement dressings. In some embodiments, as shown in FIG. 9, the thermal fluid sensor 910 may make direct contact with a skin surface 930 and may be fixed to the surface via an overlaying adhesive 920. In some embodiments, such as in FIG. 10, the thermal fluid sensor 1010 may be positioned between multiple adhesive layers 1020. In this construction, the thermal fluid sensor 1010 may be enclosed and sealed off from its surroundings providing protection from environmental ingress, such as moisture or contaminants. It will be appreciated that such a construction may be realized by integrating the thermal fluid sensor into pre-existing IV securement products. In each of these embodiments, a first section of the thermal fluid sensor may be adhered to the skin utilizing a corresponding adhesive construction and a second section of the thermal fluid sensor may extend away from the first section and is mechanically decoupled from the skin.

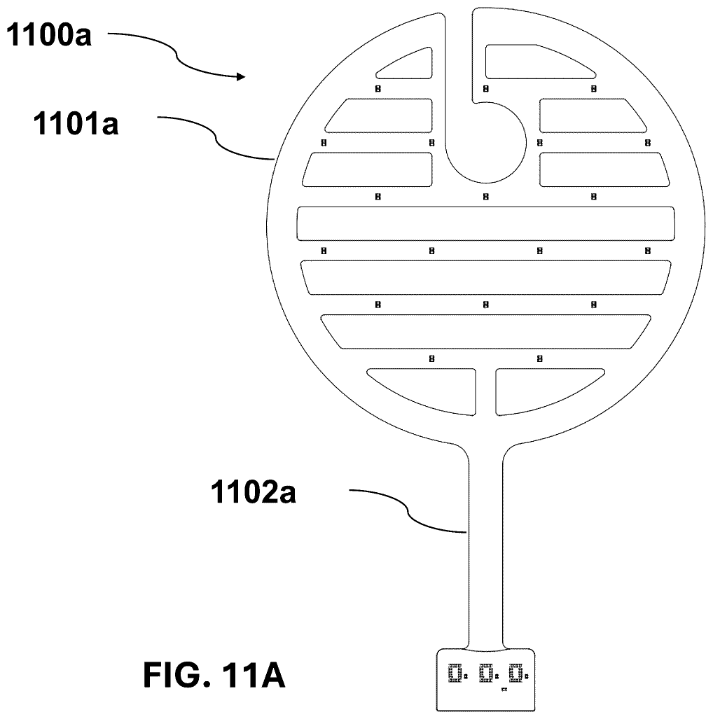

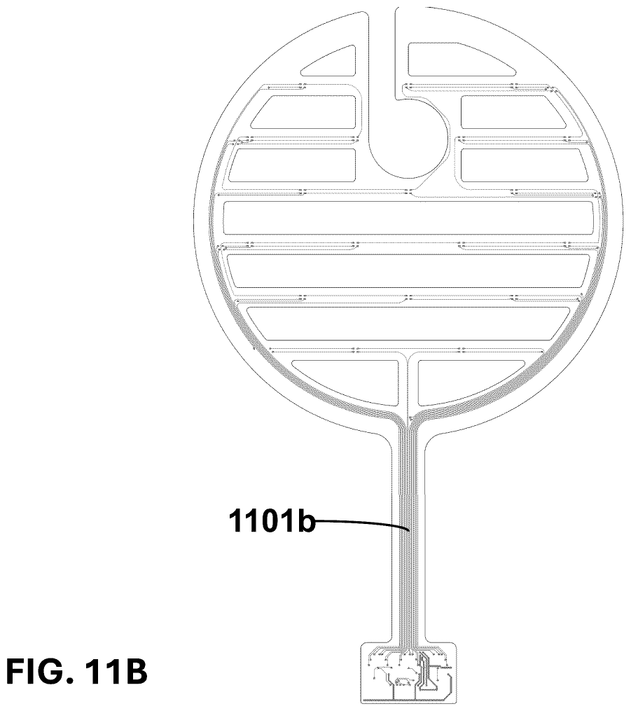

Referring to FIG. 11A, the thermal fluid sensor 1100a may be comprised of two regions, a first region 1101a containing the thermal sensing elements, and a second bridge region 1102a intended to be connected to an independent module housing additional electronic component. The relatively narrow width of the bridge region may serve to decrease the second polar moment of area of the bridge region, thereby minimizing the resistance to torsional deformation, and rotationally decoupling the first and second regions from one another, and also providing an axis of reduced bending resistance enabling improved flexibility between the regions. The length of the bridge region may reduce the shear strain enabling further rotational decoupling of the regions from one another.

FIG. 11B illustrates on example of how conductive traces 1101b may be used to transmit power and signals from sensors and/or actuators.

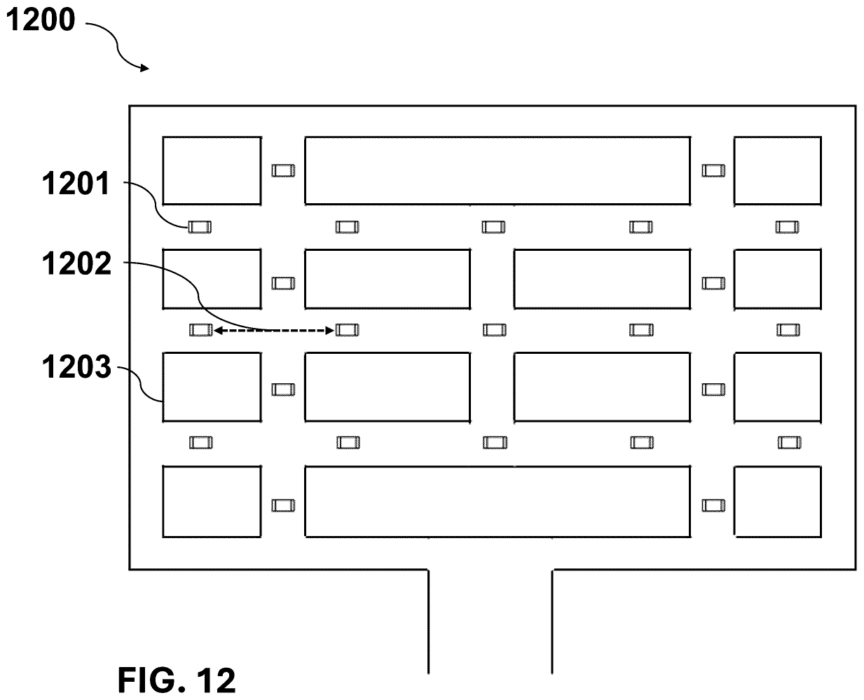

Reference is now made to FIG. 12, which illustrates an exemplary thermal fluid sensor 1200 utilizing a plurality of temperature sensing components 1201 such as, but not limited to, thermistors, negative temperature coefficient elements, positive temperature coefficient elements, resistive elements, all of which may be in the form of conventional SMT components and may be of various sizes such as 0402, 0201, or 01005, preferably 0201. These temperature sensors may be spatially arranged in a grid with each temperature sensor separated by a length L 1202 and a width W 1203 over an area A. In some embodiments, it may be advantageous for this length and width to be between 5 mm and 2 cm, preferably 1 cm. In some embodiments, the thermal fluid sensor area may be between 10 and 300 cm². These parameters may define a spatial density of temperature sensors per unit area such as 1 sensor/cm². In some embodiments, these spatial arrangements may be beneficial to optimizing a spatial extent of monitoring local to a typical IV insertion site, such as the back of a human hand or volar forearm, where the sensor may be able to identify infiltration events across several centimeters. In some embodiments, the spatial density of temperature sensors may be non-uniform across the thermal fluid sensor such that certain regions of the thermal fluid sensor may have a higher spatial density of temperature sensors. In some embodiments, these temperature sensors may be used to measure the temperatures of the underlaying skin surfaces, and these temperatures may be used to calculate and generate a thermo-spatial map of the thermal fluid sensor area. It will be appreciated that a thermal fluid sensor may be designed with a higher spatial density of temperature sensors to afford greater thermo-spatial resolution to the calculated map. As many IV infusion fluids are infused at temperatures significantly lower than body temperature such as room temperature, leakage of this fluid into the tissue surrounding the PIVC may cause evident changes in the thermo-spatial map with regions of the infiltrated tissue appearing significantly colder than adjacent regions. In some embodiments, the thermo-spatial map of the thermal fluid sensor area may be displayed on a GUI for visual monitoring.

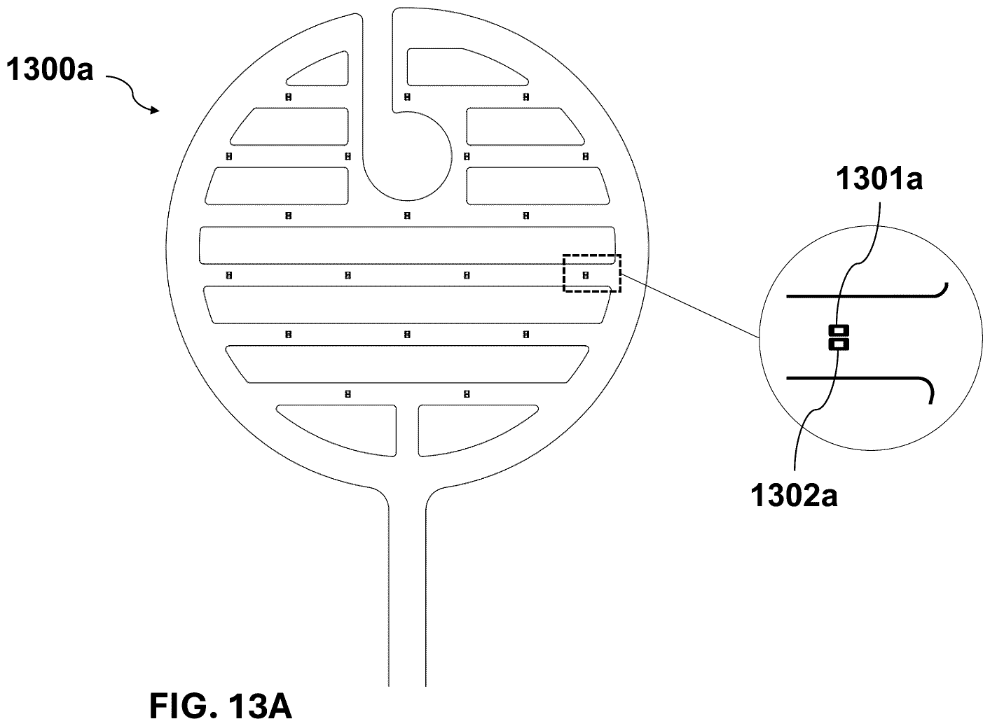

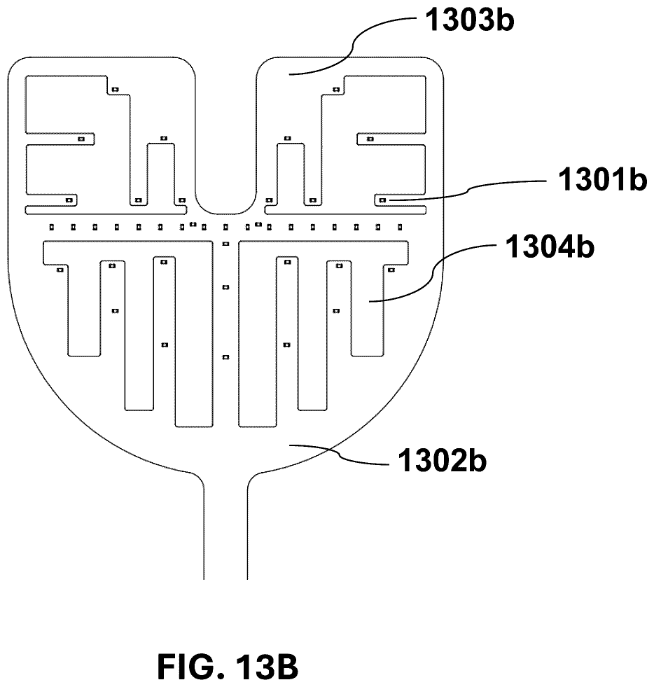

In some applications of thermal fluid sensing it is useful to utilize a plurality of temperature sensing and thermal actuating components. Referring to FIG. 13A, an exemplary thermal fluid sensor 1300a may comprise an array of pairs of temperature sensors 1301a and thermal actuators 1302a wherein the temperature sensor and thermal actuator comprising each pair are separated by a distance D and are thermally coupled. In some embodiments, each temperature sensor-thermal actuator pair will be separated by a distance L such as 1 cm over a thermal fluid sensing area. The thermal actuation elements may comprise one or more of surface mounted components such as SMT resistors (preferably 0201 sized), patterned micro-heaters, thin wire heaters, ceramic resistive heaters or polyimide-based heaters. These elements may be configured to deliver small, precisely controlled thermal power (<5 mW/mm²) to the surface of the skin or any tubing or path carrying the IV fluid, thereby creating an imperceptible local increase in temperature (˜5 K). The leakage and accumulation of fluid in the tissue surrounding the PIVC site, as is found in an infiltration event, may affect the resulting change in temperature of the surface of the skin caused by the thermal actuator as measured by the temperature sensors. As such, the thermal fluid sensor may be applied to a PIVC site to detect the onset of an infiltration event. In some embodiments, the thermal actuator may receive power as determined by a microprocessor on select intervals and the temperature sensors may record the change in temperature of the underlaying skin surface as a function of time. The thermal actuators may receive power for a time between 10 s and 60 s, such as 30 s, and the thermal actuators may be powered off for a time between 10 s and 120 s allowing the skin to return to its baseline temperature level. The microprocessor may command sequential activation and deactivation through the delivery of power to the thermal actuators under a continuous operating mode until an event is detected such as infiltration. The change in temperature in response to the selective activation of the plurality of thermal actuators as measured by the plurality of temperature sensors may be used to calculate a thermal transport parameter indicative of the state of the underlaying tissue. The calculation of this thermal transport parameter may be used as part of a set of instructions stored in the memory of the device to alert the user of an infiltration event through visual, audio, haptic, or other forms of feedback.

Referring now to FIG. 13B, in some embodiments, sensing and/or actuating elements 1301b, such as one or more temperature sensing components and thermal actuators, may be supported by a layer 1302b to provide mechanical support and electrical isolation, such as a plastic layer, such as polyimide in the commercial form of Kapton®. In some embodiments, the support layer may be a solid, continuous film in one region 1303b, while in a different device region 1304b, the layer may be patterned. The patterns may comprise regions of no support layer (e.g., patterned holes, cutouts, serpentines, or other patterns, such as greater than 25 μm thick in one region and zero thickness in other regions), or regions of varying thickness of support layer (e.g., support layer having reduced thickness in one location relative to another, such as 50 μm thick in one region and 12.5 μm thick in another).

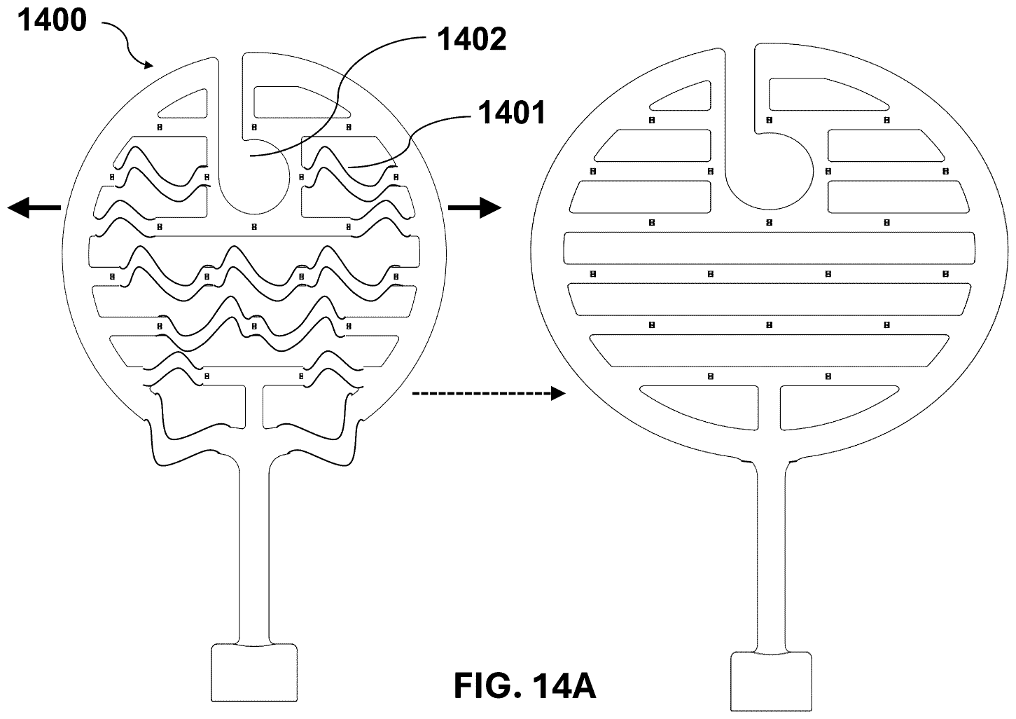

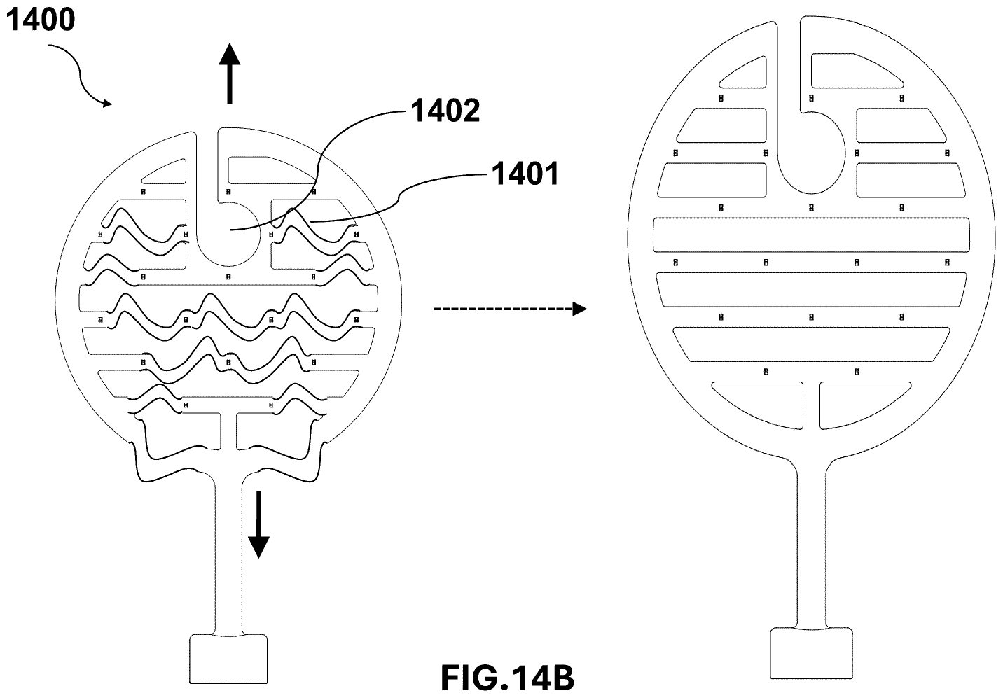

Referring to FIGS. 14A and 14B, an exemplary embodiment of a thermal fluid sensor 1400 is illustrated, featuring a support layer that is comprised of serpentine-shaped sections of Kapton® 1401. Such spatial structuring of the Kapton® provides increased device flexibility in the regions with cutouts and may also improve the ability of the device regions to conform to three dimensional surfaces. Such structuring may also facilitate conformal skin contact by enabling the device to stretch and deform as the underlaying skin surface is stretched such as during motion. In some embodiments, it may be advantageous for a sensing region with a patterned support layer to have at least 20% of the region containing no support material (such as via cutouts in Kapton®) and preferably at least 50% of the region containing no support material. Additionally, it may be advantageous for support material to be removed from regions local to individual sensors to improve mechanical conformation to surfaces, such as by having cutouts in support material on at least two sides of a sensor. In some embodiments, there may be a cutout region 1402 in the support layer to accommodate an inserted needle or catheter such that sensors may be arranged directly adjacent to and or surrounding the inserted needle or catheter.

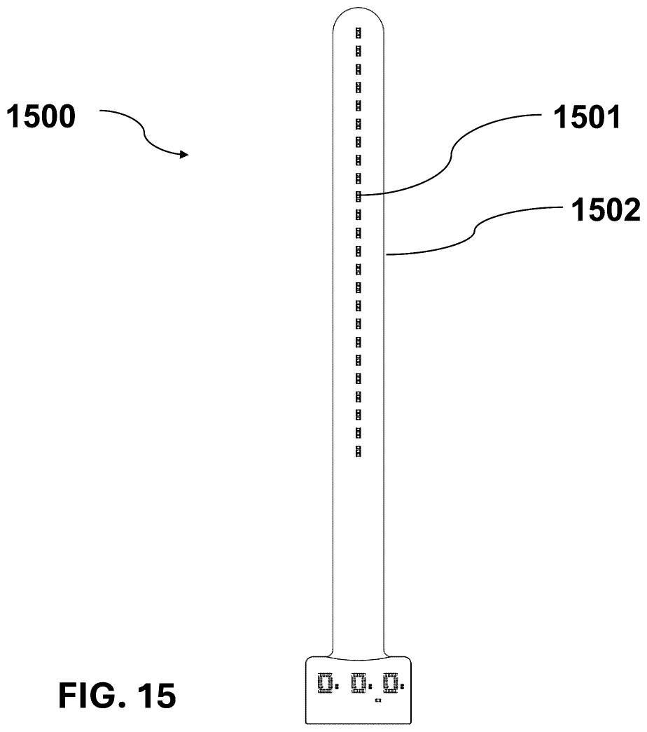

A thermal fluid sensor may be arranged in alternative ways to those described previously. Referring now to FIG. 15, one such alternative thermal fluid flow sensor 1500 may be arranged such that the thermal sensors and/or actuators 1501 and other support materials 1502 form a linear arrangement. Such an arrangement may provide increased options for placement on skin with minimal visual obstruction of the IV site of interest. Such an arrangement may adequately monitor for IV infiltration events and the like by monitoring thermal changes occurring at varying distances away from the IV insertion site, as facilitated by the linear arrangement of sensors and/or actuators.

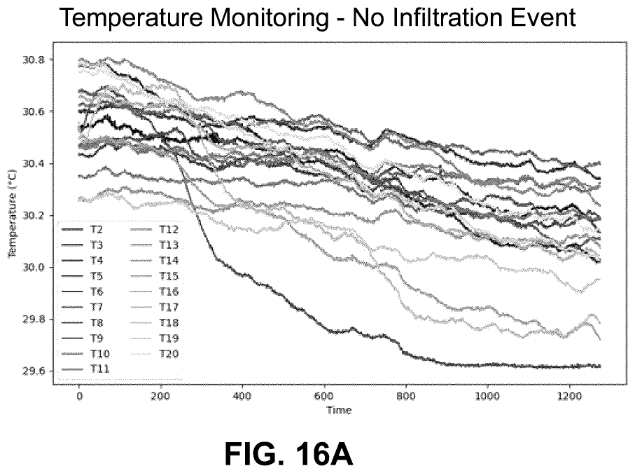

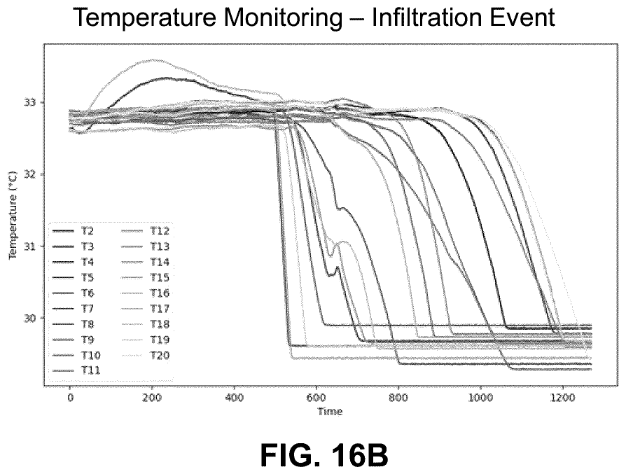

Reference is now made to FIG. 16A and FIG. 16B, which illustrate exemplary time-series temperature sensor data from each sensor in an array, consistent with some disclosed embodiments. FIG. 16A represents nominal IV flow without an infiltration event. Temperature measurements across multiple sensors remain relatively stable over a 5-minute recording time with temperature changes less than 1° C. Minor fluctuations can be observed due to the bloodstream carrying the fluid downstream or bulk colling effects, but these stay within a predictable range, reflecting the expected thermal behavior when IV fluid is properly delivered into the bloodstream without complications. In contrast, FIG. 16B highlights the temperature dynamics during an IV infiltration event. The temperature across multiple sensors drops sharply over time, indicating the leakage of cold IV fluid into the surrounding tissue rather than through the vein. The sudden and steep declines in temperature, captured by several sensors, suggest abnormal diffusion of thermal energy and synchronized cooling effect, demonstrates the ability of the device to accurately identify infiltration events in real-time.

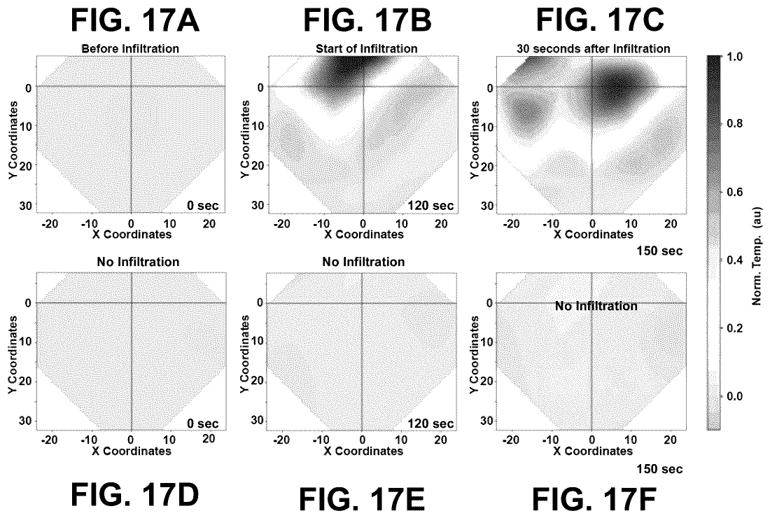

Reference is now made to FIG. 17A-F, illustrate examples of temperature data monitoring from a thermal fluid sensor associated with no infiltration event and an infiltration event, respectively, consistent with some disclosed embodiments. In some embodiments, thermal sensing units and/or heating elements are spatially defined from one another such as in previously described embodiments. These embodiments enable precise measurement of spatially mapped thermal dynamics that allow for visual understanding of PIVC complications over time, providing clinicians with valuable insights into both normal IV flow and infiltration events. As an example, shown in FIGS. 17A-C, the fluid sensor quickly identifies the change in thermal distribution between FIG. 17A and after an infiltration event has begun in FIG. 17B, expanding after an additional 30 seconds as shown in FIG. 17C, allowing for early intervention. In a different example, shown in FIG. 17 D-F, no infiltration event occurs, and the thermal distribution remains more uniform. These devices allow for visual tracking of the infiltrated IV fluid and how it spreads within the soft tissue. Such embodiments offer comprehensive monitoring of fluid infiltration, enabling clinicians to track the accumulation of IV fluids and their spread over time thus improving patient care through timely and targeted interventions.

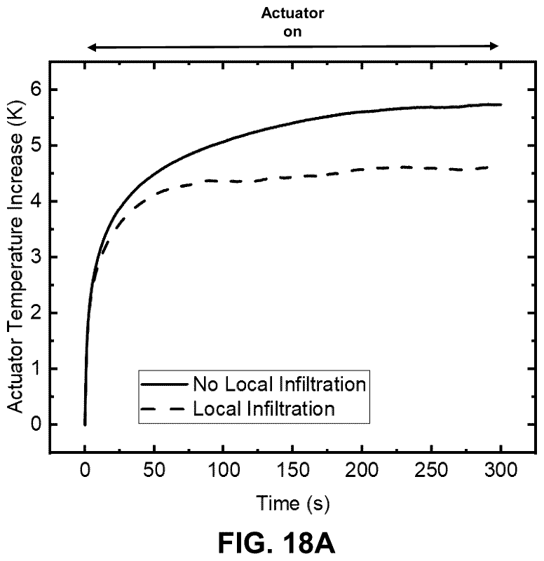

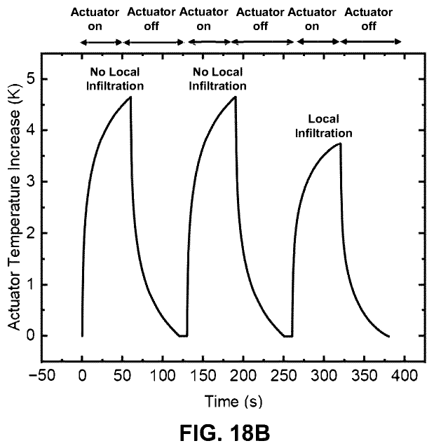

Reference is now made to FIGS. 18A and 18B, which illustrate an exemplary use of one or more thermal actuators combined with one or more temperature sensors, consistent with some disclosed embodiments. In one such example, as shown in FIG. 18A, multiple thermal actuators may be activated for an extended period, such as 300 seconds. Temperature local to the thermal actuator may be monitored by a nearby temperature sensor according to any previously described embodiment. In one such example with the thermal fluid sensor applied to skin, one actuator-sensor pair is disposed on a region without any local fluid ingress from an infiltration event (solid line) while another actuator-sensor pair is disposed on a different region where fluid has infiltrated beneath the skin (dashed line). The change in thermal properties, such as increased thermal diffusivity, due to the ingress fluid results in a lower temperature rise of the actuator for the same applied power, due to the increased ability of the local tissue to diffuse the applied heat. This figure highlights how the arrangement of thermal actuators and sensors-either co-located or spatially distributed-enables precise measurements of thermal diffusivity in tissues. In other embodiments, which may use the same actuator-sensor construction, the actuator may be activated for shorter time periods, such as 60 seconds such as in FIG. 18B, or even shorter periods such as 5 seconds.

As shown in FIG. 18B, such embodiments may enable the detection of fluid infiltration over time, such that the temperature rise of the actuator for a given cycle may be reduced once a fluid has infiltrated beneath the skin. In some embodiments, heaters may be co-located with sensors to measure localized temperature changes, while in others, they can be spaced apart in well-defined geometries to enable the calculation of thermal diffusivity using the diffusivity equation. The additional fluid absorbs and spreads heat more rapidly than surrounding tissues, enabling the system to detect infiltration and extravasation, even in cases where the IV fluids may be warmed to body temperature. In some embodiments, the system can also detect other physiological changes beneath the skin that are not necessarily temperature-dependent, such as phlebitis, where inflammation leads to altered thermal diffusivity, or ischemia, where reduced blood flow is detected through diffusivity measurements. This embodiment allows for real-time monitoring of infiltration progression and other physiological changes, supporting more effective clinical intervention.

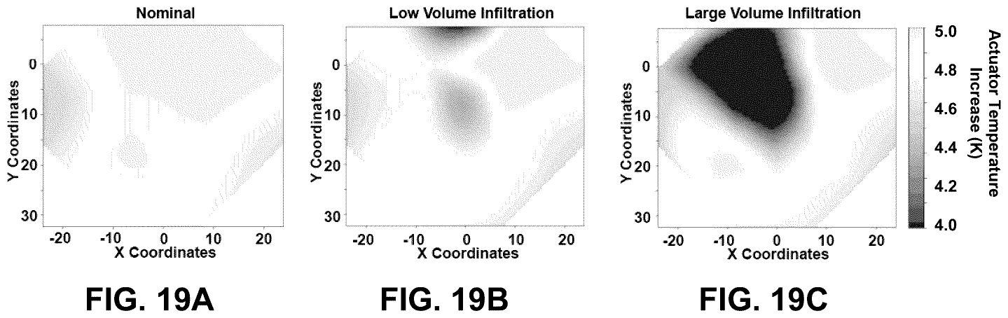

Reference is now made to FIGS. 19A-C, which illustrate an example of temperature data monitoring from a thermal fluid sensor using a thermal actuator array associated with an infiltration event, consistent with some disclosed embodiments. In some embodiments, the geometrically placed thermal sensors and actuators allow for precise monitoring of pre-warmed IV fluids that match the body's temperature. In cases where IV fluids are pre-warmed, traditional temperature measurements may not detect infiltration, as there is no difference in temperature between the IV fluid and tissue. However, by employing spatially mapped thermal sensors and actuators, local diffusivity changes caused by fluid infiltration can still be detected. In cases where temperature sensing shows no significant temperature change, the increased thermal conductivity and diffusivity from infiltrated fluid in the tissue can be accurately captured. This comprehensive understanding of fluid flow beneath the skin ensures the detection of infiltration events that would otherwise be missed with warmed IV fluids. The arrangement of thermal actuators and sensors, combined with their geometric placement for calculating thermal diffusivity enables reliable monitoring across various IV fluid therapy. Monitoring for diffusivity enables a robust device effective in all scenarios involving IV insertions.

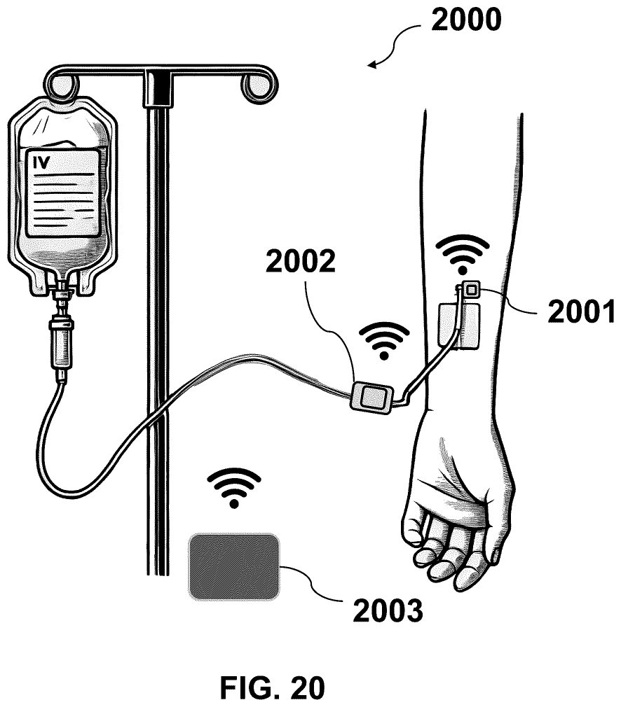

Reference is now made to FIG. 20, which illustrates a system 2000 configured to integrate the thermal fluid sensor 2001 into a broader system that extends from the patient for PIVC monitoring. In some embodiments, a second thermal actuator 2002, which can either heat using a resistive or piezoelectric system, or cool using a piezoelectric system, is positioned to control the temperature of the IV fluid. The actuator 2002 can be attached directly to the IV tubing in some embodiments and is configured to be capable of modulating the temperature of the IV fluid, either by cooling or heating it, as desired. By systematically modulating the IV fluid temperature, the system can differentiate between external environmental noise and real physiological changes, improving the accuracy of infiltration detection. To control temperature modulation, in some embodiments this system can be connected to an external system 2003, such as a smartphone or PC, for real-time control and monitoring of both the fluid temperature and any detected complications, like infiltration or extravasation. In some embodiments, the system 2000 allows the external system 2003 to be meshed with both the heater/cooler actuator 2002 and the thermal fluid sensor 2001, enabling coordinated control between multiple components. In some embodiments, the system may also mesh between the thermal actuator 2002 and the thermal fluid sensor 2001 to reduce the need for continuous connection with the PC or smartphone 2003, which may be useful for applications in diverse clinical or home-care settings. The integration of multiple components into a meshed network allows for more accurate monitoring of PIVC complications by inducing modulated temperature changes. These time-based signals help to better detect infiltration events from background noise, improving the system's reliability and detection accuracy.

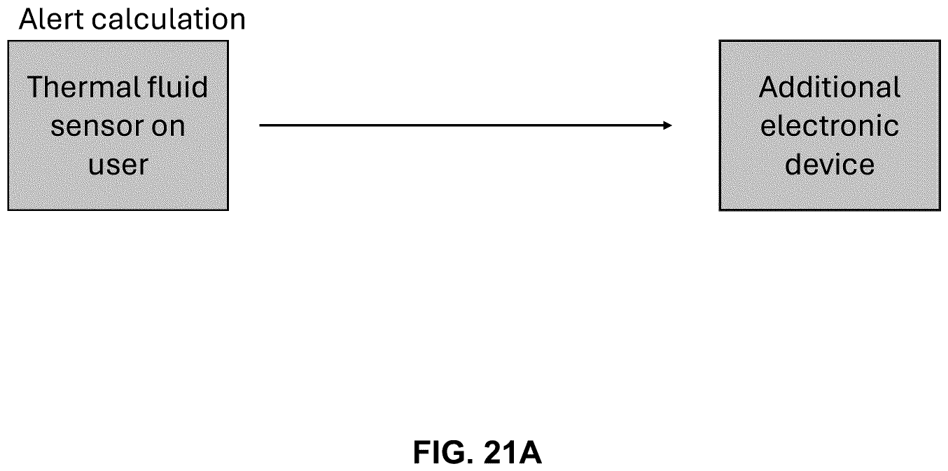

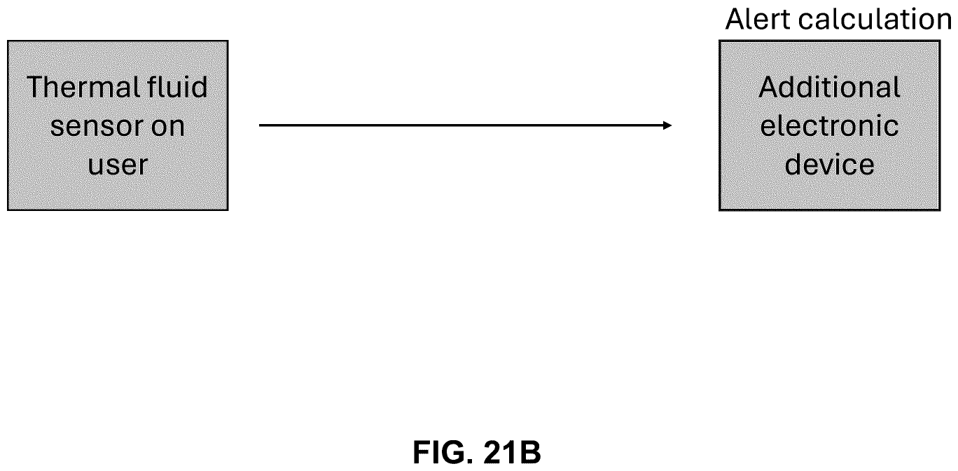

Referring now to FIG. 21A, which illustrates an exemplary thermal fluid sensing device, consistent with some disclosed embodiments. In some embodiments, thermal fluid sensing device may receive a set of signals related to the plurality of temperature sensors and calculate an alert condition based on a set of processing instructions stored in the memory storage on the thermal fluid sensor itself. The thermal fluid sensing device may alert a user through an alert notifying element found on the thermal fluid sensor such as with the flashing of an LED, transmission of a sound, or vibration of a haptic element, or other suitable methods. In some embodiment, as illustrated in FIG. 21B, the thermal fluid sensor may, alternatively or additionally, transmit this alert to an additional electronic device which may display a corresponding notification, such as on a GUI.

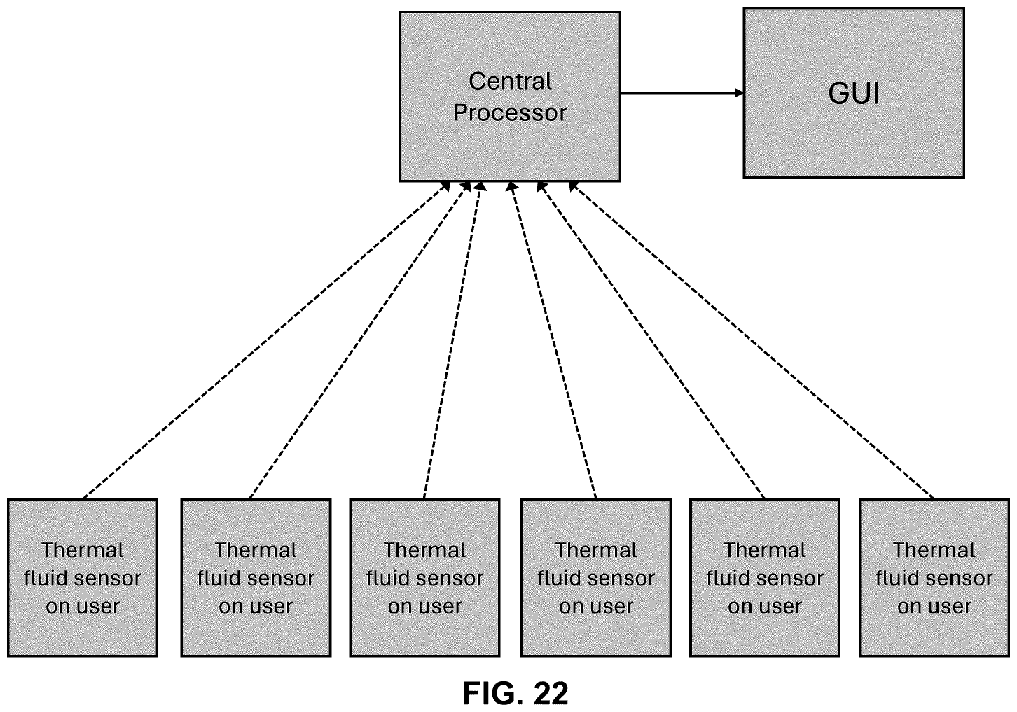

In some embodiments, such as referring to FIG. 22, the signals from the plurality of thermal fluid sensors may be transmitted such as via Bluetooth or WiFi to a separate electronic device that will process the signals, calculate an alert condition, and generate a signal to notify a user accordingly via one or more visual, audio, haptic, or other elements. In such an embodiment, a plurality of thermal fluid sensing devices may be applied to a patient population for the monitoring of an IV infiltration event. The sensors may be arranged in a network configured to process and display information about the entire patient population on a such as on a GUI of a central processing device. In such cases, clinical oversight may be realized by monitoring the GUI of the central processing device rather than monitoring each fluid sensing device individually. The central processing unit may alert a user if any one or more devices detect infiltration.

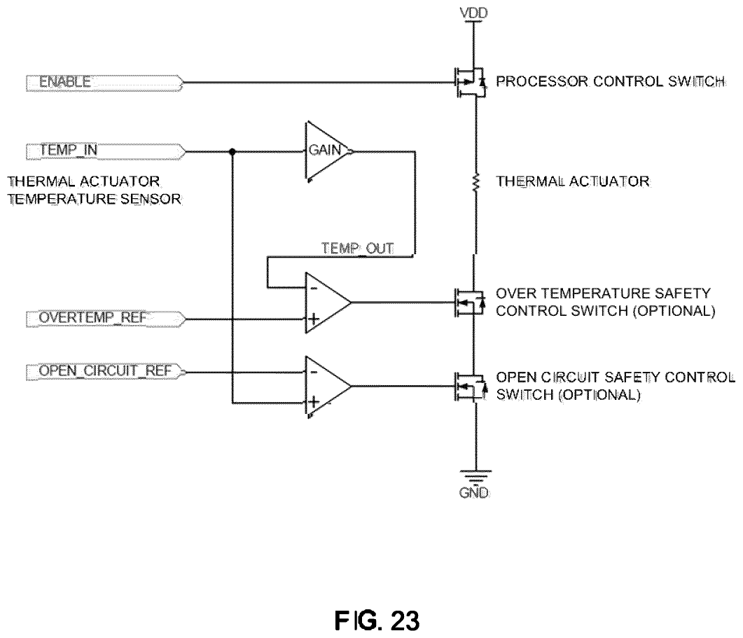

In some embodiments, it may be useful for the safety for thermal actuators to be electrically wired in an analog control circuit, such that power to the thermal actuator may be removed, via a state change of a transistor, when the temperature of the thermal actuator exceeds a threshold. Such a design, an example of which is shown in FIG. 23, may be created by wired with an analog feedback circuit such that the voltage output by a negative temperature coefficient thermistor is such that a transistor in series with the thermal actuator is turned to off when the voltage passes a threshold value. FIG. 23 additionally illustrates one such possible arrangement of an amplifier circuit used to monitor temperature of negative temperature coefficient thermistors which may be suitable in any previously described embodiment.

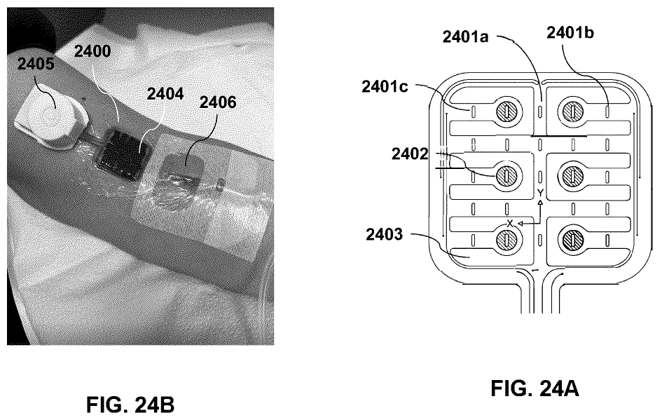

Reference is now made to FIGS. 24A and 24B, which illustrate exemplary arrangements of thermal actuators and sensors of a thermal fluid flow sensor, consistent with some disclosed embodiments. In some embodiments, a skin-mounted device 2400 may be used for detecting the infiltration or extravasation of fluids during intravenous (IV) infusion procedures. Skin-mounted device 2400 may be configured to monitor the infiltration of fluid beneath the patient's skin. In some embodiments, skin-mounted device 2400 may comprise a sensor with a plurality of temperature sensors 2401 and a plurality of thermal actuators 2402. As an example, as illustrated in FIG. 24A, skin-mounted device 2400 includes an array of temperature sensors (e.g., a 5×5 rectangular array) and an array of thermal actuators (e.g., a 3×2 rectangular array). In some embodiments, the area between the temperature sensors 2401 is at least 4 cm², and each thermal actuator may be at least 30 mm² in size. It is to be appreciated that the size of the temperature sensors or the thermal actuators may be uniform or non-uniform. It is to be further appreciated that although the plurality of temperature sensors or plurality of thermal actuators are shown as uniformly spaced, the spacing may not be limited as such. In some embodiments, skin-mounted device 2400 may include at least six temperature sensors 2401 supported by a flexible substrate. The temperature sensors 2401 may be arranged spatially to cover a region of at least 4 cm². Skin-mounted device 2400 may further include at least three thermal actuators supported by the flexible substrate. An area of a thermal actuator may be at least 30 mm² or more, 35 mm² or more, 40 mm² or more, 45 mm² or more, 50 mm² or more, 55 mm² or more, 60 mm² or more, 65 mm² or more, 70 mm² or more, 80 mm² or more, 100 mm² or more, or other suitable size.

In some embodiments, skin-mounted device 2400 comprises a flexible substrate configured to be adhered to a skin surface through an adhesive layer or an adhesive substrate. The plurality of temperature sensors 2401 and thermal actuators 2402 may be disposed on or supported by the flexible substrate. In some embodiments, skin-mounted device 2400 may include twenty-five temperature sensors 2401 and six thermal actuators 2402.

In some embodiments, the flexible substrate may comprise an active region supporting the array of temperature sensors and thermal actuators. The flexible substrate may be made of a transparent material or a substantially transparent material to allow a visual inspection of the underlying tissue. The active region of the flexible substrate is referred to as the region of the flexible substrate bound by the combined layout of the temperature sensor array and the thermal actuator array. In some embodiments, at least 20% of the active region may be transparent or substantially transparent. In a preferred embodiment, at least 30% of the active region may be transparent or substantially transparent.

FIG. 24B illustrates an exemplary skin-mountable monitoring device mounted on a human arm, consistent with some disclosed embodiments. IV infusion procedures generally involve the infusion of a fluid via a needle and an intravenous catheter 2406 into a vein located subdermally beneath the skin, typically 1-10 millimeters beneath the skin surface. Such a fluid may, in some instances, create two forms of thermal signals: 1) a change in local temperature associated with the regions beneath the skin that the infusion fluid transfers to, and 2) a change in local thermal transport properties, such as thermal diffusivity and thermal conductivity, associated with the buildup of fluid in tissue. Changes in local skin temperature may occur transiently if the infusing liquid is colder than the tissue, and may be monitored by one or more temperature sensors.

Referring back to FIG. 24A, in some embodiments, it may be desirable to employ at least six temperature sensors 2401, such that they may be disposed with at least one sensor 2401a overlying or nearly overlying the blood vessel targeted for infusion, a second sensor 2401b to the side of the vessel, a third sensor 2401c on the opposite side of the vessel, and a fourth, fifth and sixth sensor in a similar orientation at another distance along the vessel. Such an arrangement may enable a minimal level of two-dimensional spatial mapping of temperature distribution around the catheter insertion site. In some embodiments, it may be advantageous to increase the number of temperature sensors to enable a larger spatial area of monitoring or better spatial resolution. As shown in FIG. 24A, skin-mounted device 2400 may include at least 25 temperature sensors 2401 arranged in a rectangular array spaced uniformly apart.

Temperature sensing alone may not adequately resolve all infiltration events. Some infusion fluids are warmed to body temperature, reducing any thermal gradient signal created. Infiltration events may also occur deeper in the tissue, where they do not result in a significant temperature change at the surface. Finally, temperature changes are transient in nature as the fluid will quickly warm to body temperature and may be undetectable in slow infiltration conditions. As such, thermal actuators 2402 may improve capabilities for detecting infiltration of fluids by enabling the spatial assessment of changes in thermal transport properties of tissue. Such a technique may be enabled by applying power to thermal actuators with a large enough size, such as greater than 30 mm², and long enough time, such as greater than 60 seconds, to allow the heat to conduct through the tissue to the depth of the infiltration fluid, and back to other locations of the skin surface.

Referring back to FIG. 24B, it may be useful to remove sections 2403 of the device to facilitate visibility of the underlying tissue, for example such that at least 30% of total area is optically transparent. In further embodiments, it may be useful to include thermal insulation 2404, such as foam, to insulate thermal sensors from ambient effects. In yet further embodiments, the device may be rendered into a flexible, adhesive patch 2400 and integrated as part of a system to further electronics 2405 which may comprise power electronics, control electronics, communication electronics, processing electronics, memory electronics, and/or user feedback electronics.

In some embodiments, the size of skin-mounted device 2400 may be determined based on the number of temperature sensors and thermal actuators employed. For example, the size of skin-mounted device 2400 may be 0.5 sq. inches or more, 1 sq. inch or more, 2 sq. inches or more, 3 sq. inches or more, 5 sq. inches or more, 10 sq. inches or more, 12 sq. inches or more, 15 sq. inches or more, 20 sq. inches or more, 25 sq. inches or more, 30 sq. inches or more, or other suitable size.

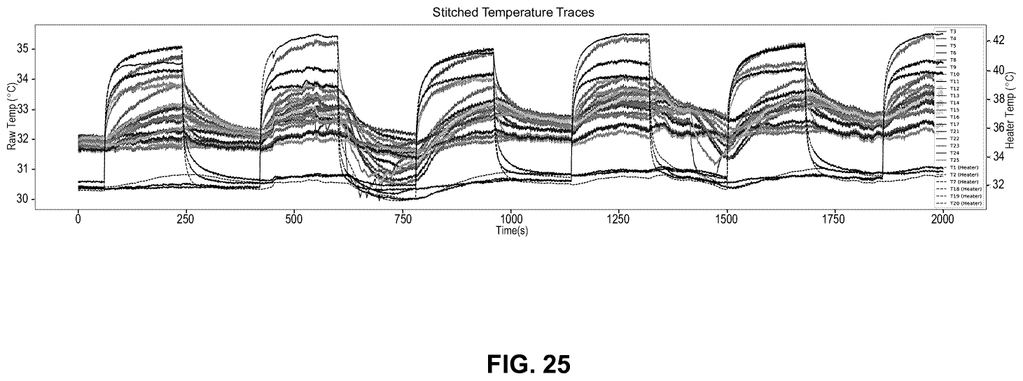

FIG. 25 illustrates examples of temperature data monitoring associated with thermal actuator use from a thermal fluid sensor, consistent with some disclosed embodiments. In some embodiments, power may be applied to all thermal actuators (e.g., heaters or thermal actuators 2402) of the skin-mounted device 2400 simultaneously. In some embodiments, power may be applied to three actuators at a time for 180 seconds at a time. In such a configuration, the temperature response to thermal actuation of each temperature sensor 2401 in the system is directly a function of the thermal properties of the tissue between the thermal actuator and the associated temperature sensor.

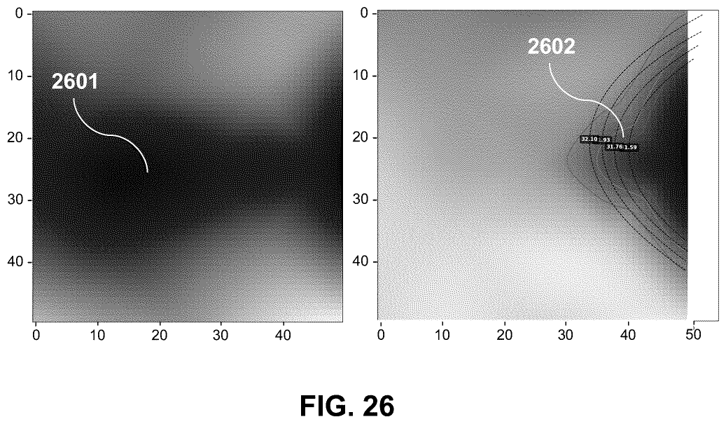

FIG. 26 illustrates examples of temperature data monitoring from a thermal fluid sensor associated with no infiltration event and an infiltration event, respectively, consistent with some disclosed embodiments. Two-dimensional spatial maps 2601 and 2602 (with interpolation between sensors) represent translation of temperature response to thermal actuation of each temperature sensor 2401. Spatial map 2601 represents the redistribution of heat in a successful infusion of fluid without infiltration, which may appear as thermal gradient along the vessel, whereas an infiltration of fluid into the surrounding tissue may result in the redistribution of fluid into an alternative shape, such as shown by spatial map 2602. Such two-dimensional maps of temperature resulting from the distribution of heat supports the use of two-dimensional temperature data to reliably identify infiltration events that may occur anywhere across the spatial area, which is an improvement over optical or impedance techniques which may target a single point. For example, one may identify the presence of an infiltration event by measuring the curvature of one or more isotherms in the spatial maps.

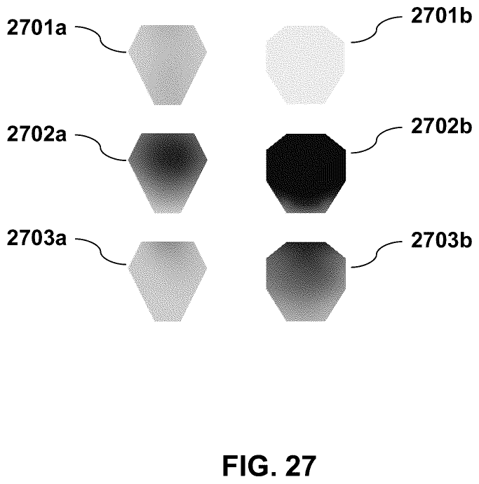

FIG. 27 illustrates examples of temperature data monitoring from a thermal fluid sensor consistent with disclosed embodiments associated with no infiltration events and infiltration events at different depths using different thermal actuator sizes. In some embodiments, using thermal actuators of spatial area less than 10 mm², the system may reliably distinguish shallow infiltration events 2702a from non-infiltration events 2701a, but may struggle to identify infiltration events 2703a deeper in the tissue. In contrast, a system with thermal actuators greater than 30 mm², may reliably identify both shallow infiltration events 2702b and deep infiltration events 2703b from non-infiltration events 2701b.

What is claimed is:

1. A system for monitoring subdermal fluid infiltration, comprising:
a flexible substrate configured to be adhered to a skin surface;
at least six temperature sensors supported by the flexible substrate and arranged spatially in a first array;
at least three thermal actuators supported by the flexible substrate and arranged spatially in a second array;
an adhesive substrate supported by the flexible substrate;
a power source configured to supply power to the at least six temperature sensors and to the at least three thermal actuators;
a memory storage device; and
a microprocessor in communication with the memory storage device and configured to:
activate the power source to supply power to at least one of the at least three thermal actuators to cause heating of a subdermal fluid;
receive information associated with a temperature of the subdermal fluid from at least one of the at least six temperature sensors;
calculate an alert condition based on a set of instructions received from a memory storage device and the temperature of the subdermal fluid; and
trigger an alert if the alert condition is met.

2. The system of claim 1, further comprising a graphical user interface (GUI) in communication with the microprocessor, the GUI configured to display a spatial map of the temperature of the subdermal fluid.

3. The system of claim 1, wherein the first array comprises a rectangular array of sixteen temperature sensors.

4. The system of claim 1, wherein the first array comprises a rectangular array of twenty-five temperature sensors.

5. The system of claim 3, wherein each of the sixteen temperature sensors is configured to detect the temperature of the subdermal fluid within a region of at least 4 cm².

6. The system of claim 4, wherein each of the twenty-five temperature sensors is configured to detect the temperature of the subdermal fluid within a region of at least 4 cm².

7. The system of claim 1, wherein the second array comprises four thermal actuators.

8. The system of claim 1, wherein the second array comprises six thermal actuators.

9. The system of claim 7, wherein an area of each of the four thermal actuators is at least 30 mm².

10. The system of claim 8, wherein an area of each of the six thermal actuators is at least 30 mm².

11. The system of claim 1, wherein the power source is configured to apply power to a thermal actuator to cause heating of the subdermal fluid for at least sixty seconds.

12. The system of claim 1, comprising twenty-five temperature sensors and six thermal actuators supported by the flexible substrate.

13. The system of claim 1, wherein at least 30% of a portion of the flexible substrate comprising the at least six temperature sensors and the at least three thermal actuators is optically transparent or substantially transparent.

14. The system of claim 2, wherein the spatial map comprises a two-dimensional temporal distribution of temperatures of the subdermal fluid.

15. The system of claim 2, wherein a calculation of the alert condition comprises a measurement of a curvature of an isotherm line in the spatial map of the temperature of the subdermal fluid.

16. The system of claim 1, wherein the at least six temperature sensors and the at least three thermal actuators are disposed on the adhesive substrate.

17. The system of claim 1, wherein the at least six temperature sensors and the at least three thermal actuators form a removable electrical connection with the power source, the microprocessor, and the memory storage device.

18. The system of claim 1, wherein the alert comprises a notification of an occurrence of an infiltration event, and wherein the notification comprises a visual notification, an audio notification, a haptic notification, or an audio-visual notification.

19. A method for monitoring subdermal fluid using a device comprising a flexible substrate, at least six temperature sensors, at least three thermal actuators, a power source, a microprocessor, and a memory storage device, the method comprising:
activating the power source to supply power to at least one of the at least three thermal actuators to cause heating of a subdermal fluid;
receiving information associated with a temperature of the subdermal fluid from at least one of the at least six temperature sensors;
calculating an alert condition based on a set of instructions received from the memory storage device and the temperature of the subdermal fluid; and
triggering an alert if the alert condition is met.

20. The method of claim 19, further comprising:
generating a spatial map of the temperature of the subdermal fluid;
displaying the spatial map on a graphical user interface (GUI) associated with the device;
measuring a curvature of an isotherm line in the spatial map; and
determining an occurrence of an infiltration of the subdermal fluid based on the spatial map.
